(19) United States
(12) Patent Application Publication
(10) Pub. No.: US 2024/0287628 A1
(43) Pub. Date: Aug. 29, 2024

(54) ALL-IN-ONE LUMINESCENCE-BASED POINT-OF-CARE TESTING DEVICE FOR VIRUS DIAGNOSIS

(71) Applicant: The Hong Kong Polytechnic University, Hong Kong (CN)

(72) Inventors: Jianhua HAO, Hong Kong (CN); Menglin SONG, Hong Kong (CN); Man Chung WONG, Hong Kong (CN); Lihua LI, Hong Kong (CN)

(21) Appl. No.: 18/173,097

(22) Filed: Feb. 23, 2023

(51) Int. Cl. C12Q 1/70; C12Q 1/6804; C12Q 1/6816

(52) U.S. Cl. CPC C12Q 1/701; C12Q 1/6804; C12Q 1/6816; G01N 2333/005

(57) ABSTRACT

This invention provides an all-in-one device for virus diagnosis, especially for a target nucleic acid sequence or target antigens or anitbodies in a single sample, comprising: a sample chamber for holding a cuvette with a UCNP-based assay and said sample; a NIR light source; a sensor for measuring fluorescence emission from a UCNP-based assay comprising a light-to-frequency converter; Optics providing an optical path ensuring only fluorescence emission from said UCNP-based assay reaches said sensor; and a microcontroller; wherein said UCNP-based assay is a system comprising UCNP surface modified with a first oligo probe and AuNP surface modified with a second oligo probe, wherein said first oligo probe and said second oligo probe are independently complimentary to separate segments on said target nucleic acid sequence that are nearby each other; or/and a system comprising ligand-free UCNP with electrostatic attraction to said target protein and AuNR surface modified with antibodies for said target protein.

### FIELD OF THE INVENTION

The present invention relates to diagnostic devices, particularly upconversion nanoparticle (UCNP)-based diagnostic device for multiple viral biomarkers (e.g. nucleic acid, antigen, antibody) detection.

### BACKGROUND OF THE INVENTION

Infectious diseases are posing an omnipresent threat to public health, especially the current outbreak of novel coronavirus disease 2019 (COVID-19) which has already infected over 540 million people and claimed more than 6 million lives worldwide as of this submission date. One of the underlying reasons for the high prevalence of these diseases is the lack of effective point-of-care detection methods. Respiratory infection virus is the most significant infection around the world and brought a terrifying threat to human beings, and its related pandemic remains volatile and is challenging to prevent. In particular, asymptomatic infected individuals of all ages can still transmit the virus to others. Containment measures including on-site and sensitive detection of viral material are an urgent key to protecting health and preventing possible spread of pandemic. Current detection methods, however, are limited by their ability to provide precise, rapid on-site diagnosis and epidemiological surveillance. For instance, traditional screening relying on conventional lateral flow antigen test suffers from inherently low sensitivity, which prohibits a relativity reliable outcome. And the quantitative reverse transcription polymerase chain reaction (RT-PCR) is the gold standard method for viral nucleic acid detection. Nevertheless, its accessibility is significantly constrained by the requirement of high-end instrumentation, multi-step reactions, special reagents, skilled personnel operating in a centralized laboratory and lengthy sample-to-answer time (˜2 h). Thus, great attention and efforts have been paid to developing novel point-of-care rapid virus detection, such as the enzyme-linked immunoassay method (ELISA), optical biosensors, and microfluidic detection. The specific proteins (spike protein, membrane protein, and nucleocapsid protein) and protease have emerged as candidate biomarkers, which provide alternative approaches for implementing preventive measures. However, sole diagnostic method cannot fully reflect all the information of infection. Considering cost effectiveness and the capability of high throughput. A point-of-care all-in-one diagnostics platform that enables synergistic detection of nucleic acid, antigen and antibody for a rapid, reliable, ultrasensitive, yet simple-to-use virus screening platform has been critical not only in the midst of the pandemic but for the stability of future, as virus will always cast a long shadow in our society. By means of all-in-one, it is either identify single virus by synergistic different biomarkers detection, or, virus identification from multiple viruses. The sooner to the identify these virus, the better placed society will be to address them. Although some on-site tests were developed in an attempt to achieve a rapid diagnosis and screening of individuals, these methods generally have no capability of achieving synergistic detection of different types of virus biomarkers let alone identifying a particular virus from one another. Moreover, a comprehensive and complementary diagnostic detection of nucleic acid, antigen and antibody provides more accurate diagnostics through reflecting the full spectrum of infected patients' information.

Fluorescent nanoprobes-based point-of-care devices have the advantages of miniaturization, ease of operation, and fast turnaround time, which facilizes them to be suitable for clinical diagnosis and offer an alternative to current nucleic detection technologies. With recent advances in nanotechnology, a myriad of luminescent nanoparticles had already been employed in various types of clinical or biological assays. Among them, lanthanide-doped upconversion nanoparticles (UCNPs) are promising nanoprobes for biodetection not only because of their superb biocompatibility. Their fluorescence mechanism permits them to be well-suited for reliable and rapid nucleic acid detection at the point-of-care. Particularly, Förster resonance energy transfer (FRET) effect can be applied to facilitate ultrasensitive detection when modified UCNPs are combined with gold nanoparticles (Au NPs). UCNPs possess low autofluorescence and exhibit a large anti-Stokes shift and minimal photodamage to genetic molecules upon near-infrared (NIR) excitation, which bestowed them with prodigious advantages over other conventional luminescent materials for point-of-care all-in-one testing. In addition. UCNPs have the merits of chemically stable, low background noises, tunable size, and resistance to photobleaching which confer them significant superiority in various biosystem detection, such as protein, microRNA, DNA, metal ions and bacteria. The energy transfer in FRET is an intermolecular dipole-dipole coupling, i.e., a very close distance (1-10 nm) from donor to acceptor. And the donor emission spectra should be overlapped with the absorption spectra of the acceptor molecule to ensure the non-radiative transition. Thus, the FRET-based detection is affected by two essential factors, i) the overlap of absorption spectra of the acceptor and emission spectra of the donor; ii) the size of the analyst corresponding to the distance between the donor and acceptor.

Inspired by the exclusive optical features of UCNPs and Au nanoparticles. In this invention, an ultrarapid, sensitive, and on-site all-in-one detection of SARS-CoV-2 RNA and Spike (S) protein based on the FRET effect between UCNPs and Au nanoparticles is developed. The main advantages of this work include (i) the high positive UCNPs are capable of capturing the free S protein and N gene efficiently, ensuring that the linker between energy excitation donor and acceptor is short enough to afford resonance between their excited states. (ii) Since conventional fluorescence-based FRET biosensors are potentially susceptible to interference with the auto-fluorescence and irrelevant pollutants in the protein samples resulting in false-positive results, the detection peak in the near-infrared (NIR) region (e.g. 800 nm) illustrates a high and specific luminescence response with negligible background interference from the sample because of the low autofluorescence and high Yb-Tm energy transfer at 800 nm. Whereas the detection of RNA can be realize with detecting 540 nm emission.

The oligo-modified lanthanide-doped core-shell UCNPs (csUCNPs) and Au NPs, are used to capture the N gene of SARS-CoV-2, demonstrating simplicity, high selectivity and sensitivity of the system. Essentially, fluorescence quenching of upconversion luminescence sandwich assay is performed, which can achieve a limit of detection (LOD) of 11.46×10⁻¹⁵ M. These exemplary performances are further optimized by structural modification of csUCNPs and Au NPs oligo-nanoprobes.

The spike (S) proteins have gained considerable interest in SARS-CoV-2 study owing to their distinct features, including that they are highly expressed on the surface of target virus and responsible for binding as well as entering into the host cells. S proteins are the primary antigens in infection and possess a more stable protein structure. Therefore, by virtue of the functional effects and structural importance of S protein, it is selected as the optimal target analyte for live virus detection.

Furthermore, portable optical diagnostic devices typically have a simple device structure consisting of an excitation source and an optical sensor. With the advancement of cheap and compact NIR light source and other essential microelectronic modules, a portable all-in-one detection device based on FRET had be devised to realize rapid synergistic and on-site screening. Further integrating the device with a smartphone not only promotes a facile read-out but also aids in uploading and sharing the diagnosis results.

Considering these merits, a point-of-care upconversion luminescence diagnostics (PULD) platform, a smartphone-controlled portable device that enables a rapid, ultrasensitive and on-site virus detection with an upconversion FRET-based assay is provided by this invention. The platform utilizes a distinctive signal detection and processing method based on current-to-frequency conversion that can significantly increase the sensor's sensitivity. The excellent quenching efficiency and detection capability of the assay are cross checked using the standard fluorescence spectrometer. Moreover, the capability of the developed PULD is further assessed using SARS-CoV-2 variant (B.1.1.529/Omicron) clinical samples and cross-validated with RT-PCR. The positive and negative results of the viral gene testing are obtained over the short turnaround time of 20 min. Significantly the results are fully agreed with the PCR testing.

The UCNP based assay for the antigen test needs only a fewer reagents, and it could be completed in minutes with the LOD of S protein as low as 1.06 fg mL⁻¹. More importantly, this design provides a new way of UCNP-based protein assay, which can be potentially expanded to the detection of related antigens, antibodies and proteins. Thus, the smartphone-controlled diagnostic platform of this invention can fulfill on-site, rapid and ultrasensitive detection of different virus biomarkers through a simple detection workflow, making the PULD a promising portable tool for rapid and direct all-in-one screening of or multiple virus biomarkers of virus or different types of viruses.

### SUMMARY OF THE INVENTION

This invention provides a portable device for detection of a target nucleic acid sequence or target protein (e.g. antibody, antigen or other protein) in a sample. In one embodiment, said portable device comprises: a) A sample chamber for holding a cuvette with a UCNP-based assay and said sample; b) A NIR light source (e.g., 980 nm) to initiate fluorescence emission from said UCNP-based assay; c) A sensor for measuring said fluorescence emission from said UCNP-based assay comprising a light-to-frequency converter: d) Optics comprising one or more filters and one or more lens, wherein said optics provides an optical path ensuring only said fluorescence emission from said UCNP-based assay reaches said sensor; e) A microcontroller for i) communicating with a controlling device; ii) determining emission intensity from said UCNP-based assay from frequency of a wavetrain from said sensor to produce detection results and sending said detection results to said controlling device; iii) implementing control instructions from said controlling device; wherein said UCNP-based assay is one or more systems selected from the group consisting of: UCNP surface modified with a first oligo probe and AuNP surface modified with a second oligo probe, wherein said first oligo probe and said second oligo probe are independently complimentary to separate segments on said target nucleic acid sequence that are nearby each other; and ligand-free UCNP with electrostatic attraction to said target protein and AuNR surface modified with antibodies for said target protein.

This invention also provides a method to detect a target nucleic acid sequence or target protein in a sample using the portable device of this invention. In one embodiment, said method comprises the steps of: a) Collecting said sample from a subject; b) Preparing said sample for said UCNP-based assay in a cuvette; c) Inserting said cuvette into said sample chamber; d) Initiating fluorescence emission from said UCNP-based assay; e) Measuring said fluorescence emission using said sensor; and f) Reading out said detection results from said controlling device.

### BRIEF DESCRIPTION OF THE FIGURES

FIG. 1 is the schematic representation of an embodiment of this invention: a point-of-care mobile phone-controlled virus diagnostic platform for multiple biomarkers detection.

FIG. 2A shows transmission electron microscopy (TEM) images of oleic acid capped core UCNPs.

FIG. 2B shows TEM images of csUCNPs.

FIG. 2C shows particle size distribution histogram of the prepared csUCNPs.

FIG. 2D shows high-resolution TEM image of csUCNPs.

FIG. 2E shows selected area electron diffraction of csUCNPs.

FIG. 2F shows upconversion emission spectra of core UCNPs and core-shell UCNPs excited by NIR laser diode.

FIG. 2G shows the P1 functionalizing process on csUCNPs.

FIG. 2H shows UV-vis absorption of P1, csUCNPs, csUCNPs-P1.

FIG. 2I shows FTIR spectra of oleate-csUCNPs, polyacrylic acid-csUCNPs, P1-csUCNPs.

FIG. 3A shows TEM image of citrate capped Au NPs.

FIG. 3B shows Modification of Au NPs with thiolated DNA via a pH-assisted and surfactant-free method.

FIG. 3C shows UV-vis absorption of P2, citrate-stabilized Au NPs, P2.

FIG. 3D shows upconversion emission spectra of core-shell UCNPs excited by NIR laser and UV-vis absorption spectra of Au NPs.

FIG. 3E shows melting curve of the sandwich assay with the N-target concentration of 20 nM.

FIG. 3F shows TEM image of hybridized duplex structure of Au NPs-Probe 1 and UCNPs-Probe 2.

FIG. 4A shows UC emission spectra of NaGdF₄:Yb/Er @NaGdF₄-probes UCNPs with various concentrations of SARS-CoV-2 oligo target in the homogeneous assay.

FIG. 4B shows the quenching efficiency with different concentrations of SARS-CoV-2 oligo target in the assay.

FIG. 4C shows the quenching efficiency of target concentrations from 200×10⁻¹⁵ M to 10×10⁻⁹ M. The relative standard deviation (RSD) was 10.38%.

FIG. 4D shows the lifetime measurement of the control sample and 200×10⁻¹² M N-target.

FIG. 4E shows the specificity test of the sandwich assay for SARS-CoV-2 virus oligo detection based on one-base (1 BM) and three-base mismatch (3 BM) gene and a complementary target at 2 nM and

FIG. 4F shows the calculated quenching efficiency according to FIG. 4E.

FIG. 5A shows the detection workflow of SARS-CoV-2 via PULD.

FIG. 5B shows the schematic of the optical and electronic components of the portable device.

FIG. 5C shows the waterfall plot of PULD read-out results for this portable platform (cutoff: 2400).

FIG. 5D shows the comparison of COVID-19 diagnostic results between the PULD and standard photoluminescence analyzer (Edinburgh Instruments FLS900).

FIG. 6 shows a schematic illustration of ultrarapid S protein detection using UCNP and AuNR system. The S protein is firstly attached to the surface of UCNPs due to high electrostatic attraction and then captured by the Au-ab NRs. Thus, an energy transfer from donor to acceptor will be occurred, and this FRET effect induces a significant decrease in upconversion luminescence.

FIG. 7A shows TEM image of NaYF₄:Yb/Tm NPs.

FIG. 7B shows high-resolution transmission electron microscopy (HRTEM) image of NaYF₄:Yb/Tm.

FIG. 7C shows scanning transmission electron microscopy (STEM) image and Na, F, Y, Yb and Tm mapping of the NPs.

FIG. 7D shows fluorescence spectrum of UCNP and absorption spectrum of AuNRs. The emission spectrum of UCNP overlaps well with the absorption spectrum of AuNRs, illustrating a promising FRET effect. The absorption spectrum was normalized at 850 nm. The upconversion luminescence spectrum was normalized at 480 nm.

FIG. 7E shows TEM image of AuNRs.

FIG. 7F shows TEM image of core-satellite structure of AuNRs and UCNPs after addition of target (S protein).

FIG. 8 shows DLS size of PEI-UCNPs and B-UCNPs.

FIG. 9 shows Zeta potential of PEI-UCNP, B-UCNP, Au NR, Au-ab NR and B-UCNP+Au-ab NR.

FIG. 10 shows the full absorption spectrum of Au NRs.

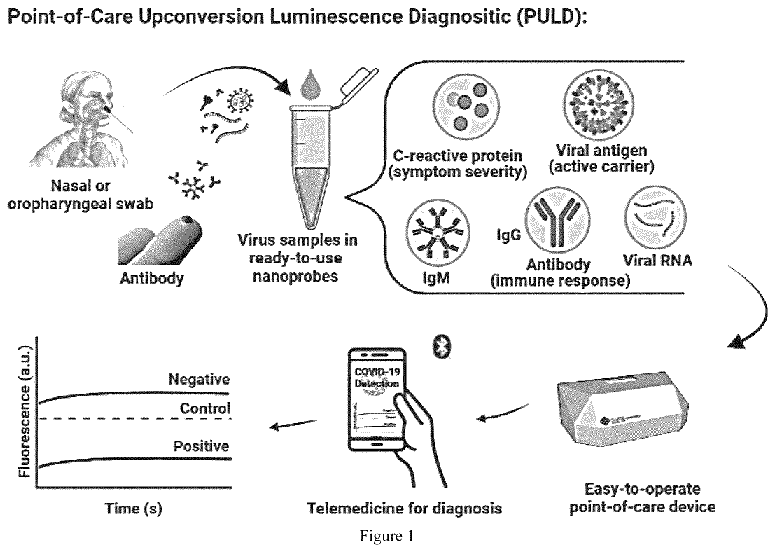

FIG. 1A shows FRET-based detection of S protein. a. Work flow of ultra-rapid detection of S protein using UCNP and AuNR based biosensors.

FIG. 11B shows UCL changes after addition of Au-ab NRs, and Au-ab NRs with S protein (1 ng mL⁴).

FIG. 11C shows lifetime changes after addition of Au-ab NRs, and Au-ab NRs with S protein at 480 nm. The marked lifetime is determined by fitting the PL decay with a single exponential function.

FIG. 11D shows lifetime changes after addition of Au-ab NRs, and Au-ab NRs with S protein at 800 nm. The marked lifetime is determined by fitting the PL decay with a single exponential function.

FIG. 12A shows UCL intensity changes with gradient S protein in swabs.

FIG. 12B shows the corresponding UCL quenching efficiency from FIG. 12A. The detection process was finished in 5 min with the optimal antibody concentration on AuNRs.

### DETAILED DESCRIPTION OF THE INVENTION

Accurate virus screening via molecular technologies is still hampered by bulky instrumentation, complicated procedure, high cost, lengthy testing time, and the need for specialized personnel. This invention provides an all-in-one point-of-care upconversion luminescence diagnostics (PULD), and a streamlined smartphone-based portable platform facilitated by a ready-to-use assay for rapid different virus biomarkers (e.g., SARS-CoV-2 nucleocapsid (N) gene, antigen, antibody and other biomarkers) testing. With the complementary oligo-modified upconversion nanoprobes and gold nanoprobes specifically hybridized with the target N gene, the FRET effect leads to a quenching of fluorescence intensity that can be detected by the easy-to-use point of care diagnostic platform. A remarkable detection limit of 11.46 fM is achieved in this diagnostic platform without the need of target amplification, demonstrating high sensitivity and signal-to-noise ratio of the assay. The capability of the developed PULD is further assessed by probing 9 RT-qPCR-validated SARS-CoV-2 variant clinical samples (B.1.1.529/Omicron) within 20 min, producing reliable diagnostic results consistent with those obtained from a standard fluorescence spectrometer. Importantly, as an example, PULD is demonstrated the capability of identifying the positive COVID-19 clinical samples with superior sensitivity and specificity, making it a promising front-line tool for rapid, high-throughput screening and infection control of COVID-19 or other infectious diseases.

In one embodiment, this invention provides a detection system consisting of upconversion nanoparticles (UCNPs) and Au nanorods (AuNRs) for an ultrasensitive, rapid, quantitative and on-site detection of SARS-CoV-2 spike (S) protein based on FRET effect. Briefly, the UCNPs capture the S protein of lysed SARS-CoV-2 in the swabs and subsequently they are bound with the anti-S antibodies modified AuNRs, resulting in significant nonradiative transitions from UCNPs (donors) to AuNRs (acceptors) at 480 nm and 800 nm, respectively. Notably, the specific recognition and quantitation of S protein can be realized in minutes at 800 nm because of the low autofluorescence and high Yb-Tm energy transfer in upconversion process. Inspiringly, the limit of detection (LOD) of the S protein can reach down to 1.06 fg ml, while the recognition of nucleocapsid protein is also comparable with a commercial test kit in a shorter time (only 5 min). This strategy is clearly technically superior to those reported point-of-care biosensors in terms of detection time, cost, and sensitivity, which paves a new avenue for future on-site all-in-one rapid viral screening and point-of-care diagnostics.

This invention provides a portable device for detection of a target nucleic acid sequence or target protein (e.g. antigen or antibody) in a single sample. In one embodiment, said portable device comprises: a) A sample chamber for holding a cuvette with a UCNP-based assay and said sample; b) A NIR light source to initiate fluorescence emission from said UCNP-based assay; c) A sensor for measuring said fluorescence emission from said UCNP-based assay comprising a light-to-frequency converter; d) Optics comprising one or more filters and one or more lens, wherein said optics provides an optical path ensuring only said fluorescence emission from said UCNP-based assay reaches said sensor; e) A microcontroller for i) communicating with a controlling device; ii) determining emission intensity from said UCNP-based assay from frequency of a wavetrain from said sensor to produce detection results and sending said detection results to said controlling device; iii) implementing control instructions from said controlling device, wherein said UCNP-based assay is one or more systems selected from the group consisting of: UCNP surface modified with a first oligo probe and AuNP surface modified with a second oligo probe, wherein said first oligo probe and said second oligo probe are independently complimentary to separate segments on said target nucleic acid sequence that are nearby each other; and ligand-free UCNP with electrostatic attraction to said target protein and AuNR surface modified with antibodies for said target protein.

In one embodiment, said NIR light source has a wavelength ranging from 700-1000 nm. In another embodiment, said NIR light source is a 980 nm light source. In a further embodiment, said NIR light source is an 808 nm light source. In one embodiment, said sensor measures said fluorescence emission at around 450 nm, 480 nm, 525 nm, 540 nm, 660 nm, or 800 nm.

In one embodiment, said microcontroller communicates with said controlling device using one or more wireless technologies selected from the group consisting of Bluetooth, WIFI, RF, and network.

In one embodiment, said controlling device is one or more selected from the group consisting of smartphone, computer, and TTL.

In one embodiment, said ligand-free UCNP or UNCP surface modified with a first oligo probe is a lanthanide-doped UCNP.

In one embodiment, said ligand-free UCNP is NaYF₄:Yb/Tm b-UCNP.

In one embodiment, said AuNR comprises: a) an aspect ratio of around 5; or b) a longitudinal absorption overlaps with emission spectra of said ligand-free UCNP in said UCNP-based assay. In another embodiment, said UCNP surface modified with a first oligo is NaGdF₄:Yb/Er@NaGdF₄ cs-UCNP.

In one embodiment, said AuNP surface modified with a second oligo has an absorbance maxima overlapping with emission of said UCNP surface modified with a first oligo.

In one embodiment, said AuNP surface modified with a second oligo and said UCNP surface modified with a second oligo are spaced less than 10 nm apart after binding to said target nucleic acid sequence.

In one embodiment, said portable device further comprises a housing for excluding external light from interfering with measurement of said fluorescence emission.

In one embodiment, said portable device has a limit-of-detection of about 1 fg mL⁻¹.

In one embodiment, a) said target nucleic acid sequence has a concentration of greater than 1 fM in said sample; or b) said target protein has a concentration of great than 1 fg mL⁻¹ in said sample.

In one embodiment, said target nucleic acid sequence or target gene is obtained from a pathogen. In another embodiment, said pathogen is one or more viruses selected from SARS-CoV-2 virus, Ebola virus, influenza virus, human immunodeficiency virus, and hepatitis virus.

In one embodiment, a) said target nucleic acid sequence is a viral gene selected from nucleocapsid. ORF1a and ORF1b; or b) said target protein is selected from the group consisting of spike protein, nucleocapsid protein, viral membrane protein, viral envelope protein and antibody against a virus.

This invention also provides a method to detect a target nucleic acid sequence or target protein in a sample using the portable device of this invention. In one embodiment, said method comprises the steps of: a) Collecting said sample from a subject; b) Preparing said sample for said UCNP-based assay in a cuvette; c) Inserting said cuvette into said sample chamber; d) Initiating fluorescence emission from said UCNP-based assay; e) Measuring said fluorescence emission using said sensor; and f) Reading out said detection results from said controlling device.

In one embodiment, said sample is collected from a source selected from the group consisting of nasal swab, oropharyngeal swab, saliva, sputum, urine, blood and feces.

In one embodiment, said step (f) is conducted after 5 to 20 minutes after step (c).

In one embodiment, said fluorescence emission is measured at around 450 nm, 480 nm, 525 nm, 540 nm, 660 nm, or 800 nm at said step (e).

The invention will be better understood by reference to the Experimental Details which follow, but those skilled in the art will readily appreciate that the specific experiments described are only for illustrative purpose and are not meant to limit the invention as described herein, which is defined by the claims that follow thereafter.

Throughout this application, various references or publications are cited. Disclosures of these references or publications in their entireties are hereby incorporated by reference into this application in order to more fully describe the state of the art to which this invention pertains. It is to be noted that the transitional term “comprising”, which is synonymous with “including”, “containing” or “characterized by”, is inclusive or open-ended and does not exclude additional, un-recited elements or method steps.

### Example 1

### Methods for Detecting Sars-Cov-2 RNA

1) Synthesis of NaGdF₄:Yb/Er Core UCNPs (cUCNPs)

The UCNPs were synthesized by layer-by-layer epitaxial growth. Acetate Ln³⁺ solutions (Ln³⁺=Gd³⁺, Yb³⁺ and Er³⁺, 0.4 mmol) in a 50 mL three-neck flask with identified molar ratio were introduced to a 50 mL three-neck flask. Next, octadecene (ODE) (6 mL) and oleic acid (OA) (4 mL) were added to the flask under magnetic stirring. After heating to 150° C. for 40 minutes, the mantle was removed, and the mixture was cooled to room temperature. Afterwards, NaOH/methanol (1 mmol) and NH₄F/methanol (1.32 mmol) were thoroughly mixed and added to the reaction mixture. The temperature was maintained at 50° C. for 30 minutes with vigorous stirring. Following degassing in vacuum for 15 minutes, the flask was heated to 285° C. for 1 hour and 10 minutes under argon gas protection. UCNPs were collected by centrifugation and purified with cyclohexane and ethanol after cooling to room temperature. Lastly, the UCNPs were dispersed in the cyclohexane for further use.

2) Synthesis of NaGdF₄:Yb/Er, @NaGdF₄ Core-Shell UCNPs (csUCNPs)

0.4 mmol of acetate of Gd³⁺ was added to a 50 mL three-necked flask. Then, OA (4 mL) and ODE (6 mL) were added to the flask while magnetically stirring. After heating to 150° C. for 40 minutes, the mixture was cooled to room temperature. Under vigorous magnetic stirring, the as-prepared UCNPs were then introduced into the Ln³⁺ precursors. Then, a mixture of NaOH/methanol (1 mmol) and NH₄F/methanol (1.32 mmol) was prepared and immediately added to the reaction mixture. Afterwards, the temperature was held at 50° C. for 30 minutes. After degassing the flask for 15 minutes in vacuum, the temperature was raised under the protection of argon gas to 285° C. in 1 hour and 10 minutes. Crude csUCNPs were centrifuged after cooling to room temperature and purified with cyclohexane and ethanol. Finally, the csUCNPs were dispersed in cyclohexane to undergo further modifications.

### 3) Synthesis of 5 nm Au NPs

Citrate-stabilized AuNPs were synthesized based on a previous study. Briefly, 0.1 mL of tetrachloroauric acid (HAuCl₄, 25 mM) was injected into a mixed solution containing 15 mL of sodium citrate (SC, 2.2 mM), 0.01 mL of tannic acid (TA, 2.5 mM) and 0.1 mL of K₂CO₃ (150 mM) at 70° C. for 5 minutes under vigorous stirring. Then, two additional shots of HAuCl₄ (0.05 mL, 25 mM) were added every 10 minutes to the medium. Afterwards, the citrate stabilized AuNPs were obtained and stored at 4° C. for further modification.

4) Preparation of Ligand-Free csUCNPs

To prepare the ligand-free NaGdF₄:Yb/Er@NaGdF₄ csUCNPs, the oleate-capped UCNPs were removed by acid treatment. Typically, the as-synthesized oleate-capped UCNPs were dispersed in 15 mL of acidic ethanol solution (pH=1, prepared by mixing 100 μL of concentrated hydrochloric acid with 14.9 mL of absolute ethanol) and ultrasonically treated for 40 minutes to remove the surface ligands. Then, the UCNPs were collected by centrifugation at 4000 rpm for 30 minutes, followed by further purification with an acidic ethanol solution (pH=4, obtained by adding 10 μL of concentrated hydrochloric acid with 14.9) mL of absolute ethanol). Afterwards, the products were washed three times with ethanol and deionized water, followed by a redispersion in 1 mL deionized water.

### 5) Au NPs-Oligos (P2) Conjugation

10 nmol thiol modified oligonucleotide (P2) was treated with 10 μL DTT (0.1 M) for 30 minutes to cleave disulfide bond. According to the manufacturer's instructions, the activated oligonucleotides were purified using gel columns (illustra MicroSpin G-25 Columns, GE Healthcare, UK) and characterized by UV-vis spectrophotometer (Shimadzu, Japan). As previously reported, the AuNPs were then subjected to instantaneous oligo modification by adding DTT-treated P2 (0.1 M, 10 μL) to citrate-stabilized AuNPs (800 μL), followed by the addition of PBS buffer (4 μL, 50 mM) and citrate buffer (10 μL). After incubation for 10 minutes, the AuNPs-P2 were obtained by high-speed centrifugation. This step was repeated three times, after which AuNPs-P2 was dispersed in 1 mL of RNase-free water.

### 5) Hybridization of Oligos

After diluting the stock solution (100 μM), a variety of concentrations of the N target of SARS-CoV-2 was prepared (200 μM, 50 μM, 20 μM, 2 μM, and 200 fM). As a typical hybridization procedure, N-target was mixed with AuNPs-P1 (200 μL) and csUCNPs-P2 (100 μL) in PBS buffer for 40 minutes at 45° C. in a heating block. From UCL spectra, the quenching efficiency (ηx) of Au NPs on UCL can be calculated by

$${η}_{x} = \frac{{I}_{0} - {I}_{x}}{{I}_{0}} \qquad ( 1 )$$

It should be noted that I₀ is the upconversion luminescence intensity of csUCNPs and 1ₓ is the upconversion luminescence intensity after adding the Au NPs-probe2 in combination with different concentrations of target RNA.

### 7) Upconversion Luminescence Spectra and Lifetime Measurement

The upconversion luminescence spectra were measured after cooling the samples to room temperature using a NIR diode laser as an excitation source, and then recorded by an Edinburgh Instruments FLS920 fluorescence spectrometer. A total of three measurement steps were performed to get the average intensity of each concentration. The luminescence lifetime of the mixture of UCNPs and Au NPs and Au NPs-P1-target-P2-UCNPs were measured by Edinburgh FLS 900 coupled with DG-535 digital pulse generator and NIR diode laser (modulation mode, Changchun New Industries Optoelectronics Technology Co., Ltd.).

### 9) FRET Efficiency Calculation

According to Theodor Forster's quantitative theory, nonradiative energy transfers depend on the spectral overlap and the intermolecular distance. Energy transfer rate was calculated as the integral of spectral overlap. Here is the simplified equation for FRET efficiency:

$$E = \frac{1}{1 + {( \frac{R}{{R}_{0}} )}^{6}} \qquad ( 2 )$$

where R represents the distance between donors and acceptors, and R₀ represents the fluorescent distance between donors and acceptors when the transfer efficiency is 50%. The energy transfer efficiency (E) is inversely proportional to the sixth power of the distance between donors and acceptors. With a decreasing distance R, the transfer rate is about to reach its maximum (less than R₀). In addition, R₀ could be calculated to be 10-100 Å using overlap integrals, which is consistent with previous experiments. Therefore, by measuring E and calculating R₀, the molecular distance between donors and acceptors can be determined.

### 10) Details of PULD Device

The PULD portable device was modeled in a computer-aided design software (Solidworks, 2018) and the housing of the device was fabricated via a Fused Deposition Modeling 3D printer (Creatly, Ender 3v2) extruding thermoplastic Polylactic Acid (PLA) filaments. The compact dimension of the PULD device (15.0×6.0×6.7 cm³) not only served as a dark chamber to elude external light from interfering with the fluorescence measurements, but also enclosed all the optical and optoelectronic components. The device is driven by a 12 V Li-Po battery. The light sources partaking in the measurement for fluorescence excitation is an infrared LED. The LED is driven by a 3.3 V buck convertor and a constant current amplifier (Texas Instrument, LM358), is coupled with a convex focusing lens and attached to a metal heatsink. The detector subassembly for detecting fluorescent signals from the sample chamber consists of an in-house printed cuvette holder, two bandpass filters (FUZHE, FU-550ZDLGP-F5*5 and FU-532LGP-Y12*0.7-700 nm), a bandpass plano-convex lens (HY optic, GCL-010130, f 6), and an ultrasensitive light-to-frequency converter (ams, TSL237) function as a sensor. All these optical components are assembled firmly in the PULD device thus they are concentrically and precisely aligned inside the housing. More importantly, a thorough investigation was conducted to ensure the excitation electromagnetic wave posed no interference onto the measurement result. The light from the infrared-LED luminescence is used as an excitation source for the assay. Fluorescence signals from the cuvette were then recorded by the photodiode in the sensor and converted to a square wave by the monolithic current to frequency converter. An Interrupt Service Routine was implemented in a C++ program and uploaded to a microcontroller board (Arduino nano, ATmega 328P) inside the PULD device to coordinate the screening process. These measured values were transmitted in real-time to a smart phone paired with the microcontroller board though a Bluetooth connection (module: HC-06). An in-house APP was developed to control the PULD device and present the screening result.

### 11) Simple and Rapid Testing Procedure for PULD

The intensity of the fluorescence emission from the sample was measured by the sensor in PULD, the microcontroller recorded the corresponding values. In turn, the C++ program in the microcontroller requires resolution and accuracy of the detection by pulse accumulation and appropriate integration times to eliminate jitter in the noise. The simple screening procedure begins with inserting a cut-off value or a cuvette with only oligonucleotide modified probes as control into the PULD device. The control value was measured from the control sample and recorded in the microcontroller while simultaneously displayed on the smartphone. Upon changing the control with the real sample, measurement was then taken and recorded. By comparing the control's value and the sample's value, the screening results were obtained and displayed on the user's smartphone. The whole screening procedure takes around 1 to 2 minutes.

### Example 2

### Detection of Viral RNA

### 1) Design of PULD for Multiple Virus Biomarkers Detection

The designed rapid and point-of-care virus diagnostic system should be capable of providing a simple workflow, therefore this invention developed an easy-to-operate diagnostic platform for virus screening by adopting a one-pot detection protocol with an effective upconversion luminescence sandwich assay and a smartphone-controlled portable device. This diagnostic system is termed as PULD. As shown in FIG. 1, UC-FRET bioprobes can be conjugated with various bio-recognition elements for multiple biomarkers detection to realize multi-level analysis The resultant upconversion quenching due to FRET effect is recorded by the portable device and the results are presented to the user through a smartphone via Bluetooth, thus enabling fast and accurate viral RNA, or antigen or antibody detection.

### 2) PULD for RNA Detection

More specifically, Au NPs were conjugated with thiol-modified oligonucleotides, whereas polyacrylic acid-modified csUCNPs were conjugated with amine-modified oligonucleotides. In the presence of the SARS-CoV-2 N gene, oligonucleotide hybridization between complementary pairs occurs, bringing csUCNPs and Au NPs into proximity. Under the excitation of NIR, the fluorescence of csUCNPs can be absorbed by Au NPs due to FRET effect. Thereby, the N gene of SARS-CoV-2 is quantified and screened by measuring the variation of the fluorescence intensity. Benefiting from the superior sensitivity and specificity of the FRET-based sandwich assay in direct and rapid detection of the SARS-CoV-2 Omicron variant, this invention designed and customized a low-cost smartphone-controlled device to demonstrate its practicality and accessibility. Signals were recorded by the microcontroller (Arduino Nano) in the device that also coordinated viral RNA detection and transmission of detection data to the user's smartphone via Bluetooth connection. The screening results were subsequently displayed in the smartphone when the recorded values had been processed by the microcontroller. The detection setup of the portable device allows for ultrasensitive fluorescence detection. Firstly, a 500 μL sample cell containing clinic sample was introduced into the chamber. The screening test was initiated by the user via Bluetooth connection between mobile phone and the microcontroller. When the NIR excitation beam was turned on, fluorescent signal was then recorded by a photodiode integrated with a current to frequency converter composed of an operational amplifier integrator was incorporated into the photodiode to enhance the detection sensitivity. The detected signals were recorded by the microcontroller to determine the outcome of the screening. Finally, the test results were presented to the user's smart phone. For the selective detection of SARS-CoV-2 isolate 2019-nCoV/USA-WA1-A12/2020, N gene was selected as a well-conserved target since it was able to demonstrate organism-specificity with a sufficient distinction from related species. A set of antisense oligonucleotides were predicted by following the methodology described in Materials and Methods. According to their binding disruption energies and binding energies with the target sequence, one of the predicted antisense oligonucleotide sequences was selected (Table 1). Moreover, to achieve effective probe conjugation to nanoparticles and hybridization, the oligo probes were attached to poly-A bases. Design of the oligo probes for the target sequences is based on S fold software analysis. The oligonucleotides used in this work are listed in Table 2.

A Sequence Listing XML file containing SEQ ID No.: 1-19, created on Feb. 23, 2023 with a file name of 3576-US.xml and having a size of 21 kb, is hereby incorporated-by-reference into this specification.

TABLE 1
Selected antisense oligo (5p→3P) sequences targeting N gene of SARS-CoV-2.

| Starting | | | | Average | | |
|---|---|---|---|---|---|---|
| target | | | | unpaired | Binding site | Oligo |
| position - | | | | probability for | disruption | binding |
| ending target | Target sequence | Antisense oligo | | target site | energy | energy |
| position | (5p - 3p) | (5p - 3p) | GC content | nucleotides | (kcal/mol) | (kcal/mol) |
| 421 → 440 | ACACCAAAAG | CCAATGTGAT | 40.0% | 0.787 | 7.7 | −15.4 |
| | AUCACAUUGG | CTTTTGGTGT | | | | |
| | (SEQ ID NO.: 1) | (SEQ ID NO.: 6) | | | | |
| 443 → 462 | CCCGCAAUCC | ATTGTTAGCA | 50.0% | 0.651 | 7.6 | −10.2 |
| | UGCUAACAAU | GGATTGCGGG | | | | |
| | (SEQ ID NO.: 2) | (SEQ ID NO.: 7) | | | | |
| 799 → 818 | GCAUACAAUG | GCTTGTGTTAC | 40.0% | 0.749 | 5.4 | −12.9 |
| | UAACACAAGC | ATTGTATGC | | | | |
| | (SEQ ID NO.: 3) | (SEQ ID NO.: 8) | | | | |
| 836 → 855 | CAGAACAAAC | ATTTCCTTGGG | 40.0% | 0.708 | 5.8 | −14.4 |
| | CCAAGGAAAU | TTTGTTCTG | | | | |
| | (SEQ ID NO.: 4) | (SEQ ID NO.: 9) | | | | |
| 886 → 905 | ACUGAUUACA | GGCCAATGTTT | 40.0% | 0.694 | 8.5 | −10.0 |
| | AACAUUGGCC (SEQ | GTAATCAGT (SEQ | | | | |
| | ID NO.: 5) | ID NO.: 10) | | | | |

TABLE 2
The oligo probes and target sequences were used in
this study, the Bold text indicates non-
complementary bases.

| Name | Sequence (5p → 3p) |
|---|---|
| Probe 1 | ATTTCCTTGGAAA-NH₂ (SEQ ID NO.: 11) |
| Probe 2 | SH-AAAGTTTGTTCTG (SEQ ID NO.: 12) |
| 3 BM | CAGAAGAAACGCAAGCAAAT (SEQ ID NO.: 13) |
| target | |
| 1 BM | CAGAAGAAACGCAAGCAAAT (SEQ ID NO.: 14) |
| target | |
| N-target | CAGAACAAAC CCAAGGAAAT (SEQ ID NO.: 15) |
| N-f | GGGGAACTTCTCCTGCTAGAAT (SEQ ID NO.: 16) |
| primer | |
| N-r | CAGACATTTTGCTCTCAAGCTG (SEQ ID NO.: 17) |
| primer | |
| RT-qPCR | FAM-TTGCTGCTGCTTGACAGATT-TAMRA (SEQ ID |
| probe | NO.: 18) |
| N-cDNA | GGGGAACTTCTCCTGCTAGAATGGCTGGCAATGGCGGTG |
| | ATGCTGCTCTTGCTTTGCTGCTGCTTGACAGATTGAACC |
| | AGCTTGAGAGCAAAATGTCTG (SEQ ID NO.: 19) |

3) Structural and Optical Properties of csUCNPs Modified with Oligo Probes

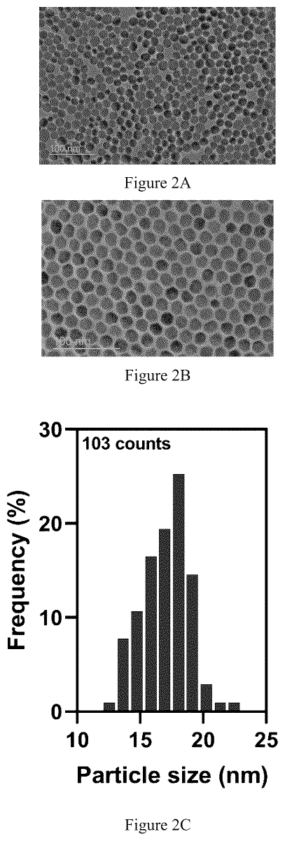

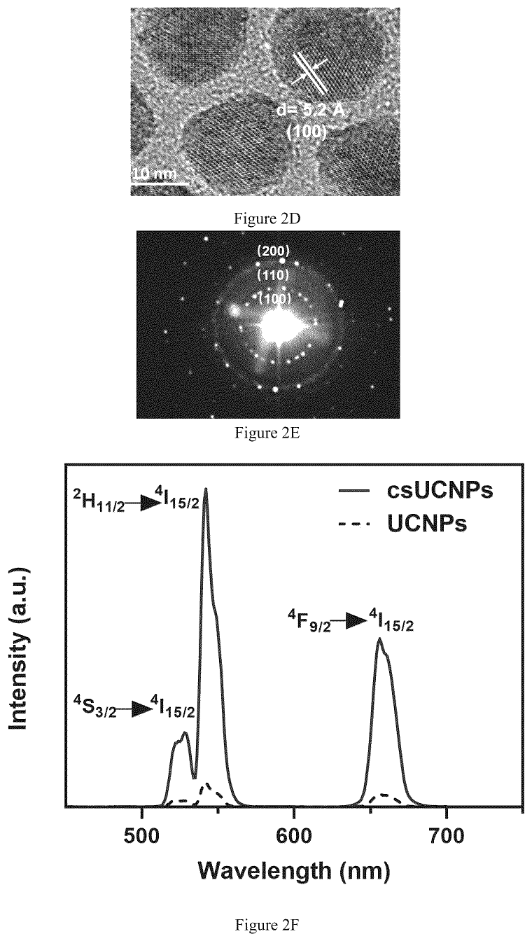

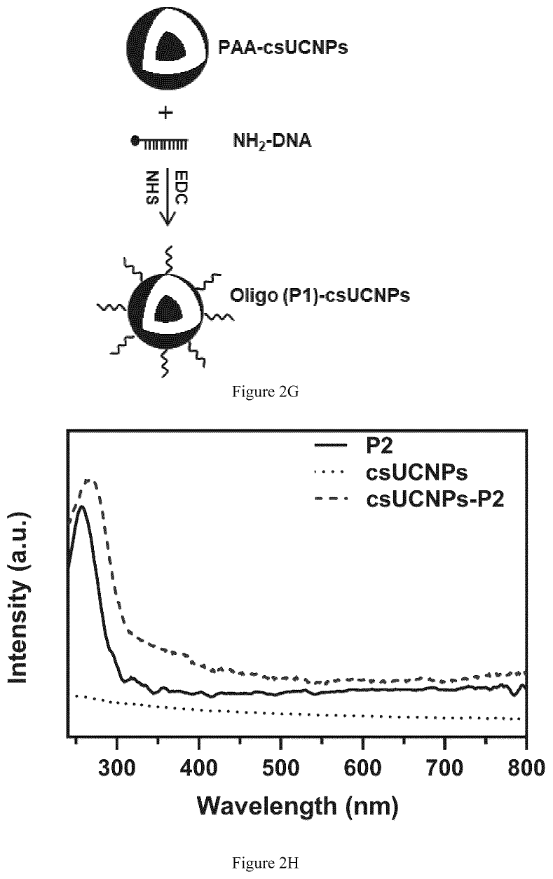

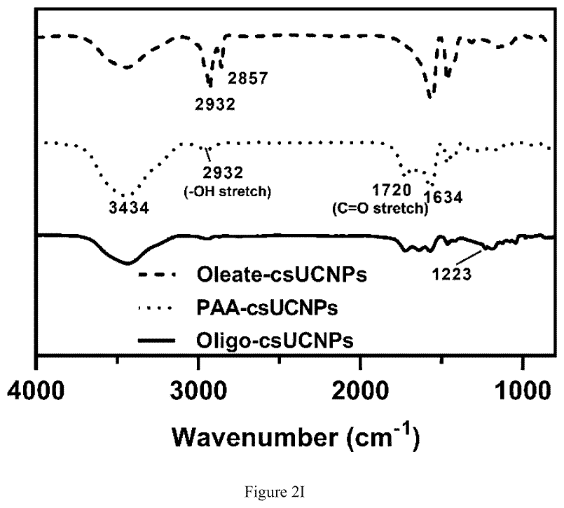

The core NaGdF₄:Yb/Er nanoparticles with an average diameter of 16 nm were synthesized through co-precipitation strategy in a binary solution of oleic acid and 1-octadecene. (FIG. 2A). To enhance the fluorescent intensity for a more sensitive detection, the UCNPs were further modified to form a core-shell structure. Specifically, similar to the preparation of the NaGdF₄, NaGdF₄: Yb³⁺/Er³⁺@NaGdF₄ core-shell structure can be obtained by adding core nanoparticles to the precursor solution as seed crystals prior to adding the precipitator. The corresponding transmission electron microscope (TEM) images of Er³⁺-doped core-shell UCNPs are illustrated in FIG. 2B, revealing a uniform morphology with an average size of 18 nm (FIG. 2C). A high-resolution TEM image depicted the single crystalline nature of the core-shell nanocrystals, as exhibited by clear lattice fringes spaced at 0.52 nm (FIG. 2D), which agrees with the lattice spacing in planes (100) of hexagonal-phase NaGdF₄. As shown in FIG. 2E, the selected area electron diffraction (SAED) pattern represents the polycrystalline diffraction rings associated with the specific planes of the hexagonal NaGdF₄ lattice It is a fact that the size of the acceptor can determine the FRET distance as well as the spatial conditions of initial contact, zipping, and stability during hybridization. The emission spectrum of the csUCNPs under the excitation of NIR is depicted in FIG. 2F. Two UC bands were observed at 520/540 nm and 654 nm, corresponding to the ²H_{11/2}/⁴S_{3/2}→⁴I_{15/2} and ⁴_{15/2} transitions of Er³⁺, respectively. The visible UC emissions from NaGdF₄:Yb³⁺/Er³⁺ in hexagonal phase were enhanced by 12.8 times because of the growth of a thin layer of NaGdF₄ (˜2 nm). The core-shell structure can effectively suppress surface-related deactivations and spatially isolate the core nanoparticle from other deactivators (ligands, solvents, etc.). Importantly, in the design of this invention, the oligo-modification is a vital step not only for capturing N gene of SARS-CoV-2 during the detection but also for facilitating the FRET effect to produce fluorescence intensity variation. Precisely, csUCNPs modified with polyacrylic acid (PAA) were prepared for bioconjugation of the oligo probe via coordination interaction between carboxylic groups and lanthanides ions. Afterwards, the oligo probe was conjugated to the carboxylic acid terminals using covalent crosslinking of carbodiimide and n-hydroxysuccinimide (EDC/NHS) (FIG. 2G). It is worth noting that the oligonucleotides have a characteristic peak of 260 nm in the UV-vis absorption spectrum. This invention made use of this feature to confirm whether cDNAs were successfully modified on csUCNPs. As shown in FIG. 2H, a sharp peak at around 265 nm after oligo-conjugation under UV-Vis absorption investigation, which indicates functionalization of P1 on csUCNPs. Furthermore, Fourier-transform infrared spectroscopy (FTIR) spectra of the oleate, PAA and oligo capped UCNPs are shown in FIG. 2I. It is evident that the CH₂ groups have been successfully removed from the layer of oleate by considering the disappearance of the major peaks at 2939 and 2857 cm⁻¹. Besides, the carbonyl stretch of a carboxylic acid C═O appears as a very intense band at 1720 cm⁻¹ while the carboxylic acid O-H stretch appears as a very broad band at 2932 cm⁻¹ after PAA modification. In particular, the wavenumber at 1223 cm⁻¹ relevant to the oligo represents the vibration of symmetric phosphate (PO₂), which confirms the conjugation of oligo onto the csUCNPs.

4) Structural Properties of Au NPs and Modification with Oligo Probes

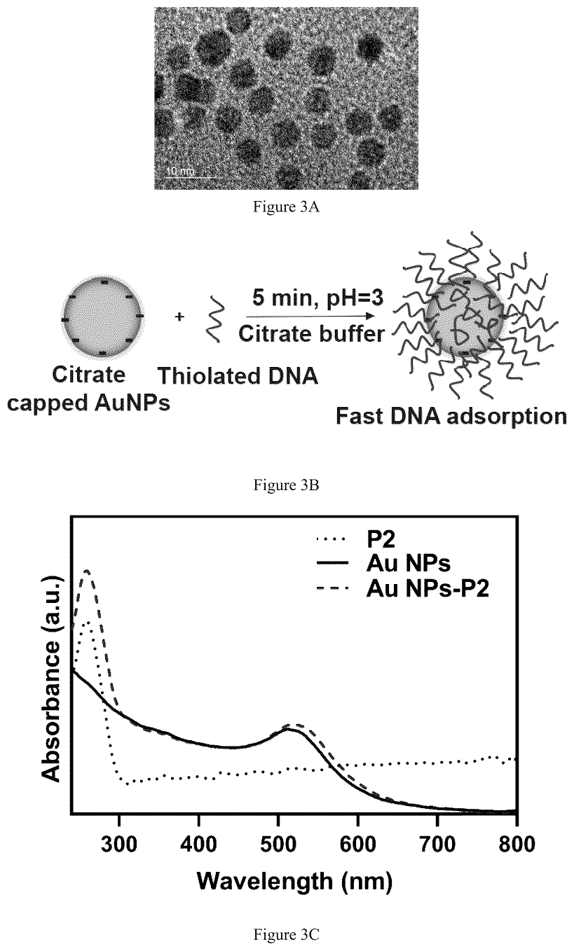

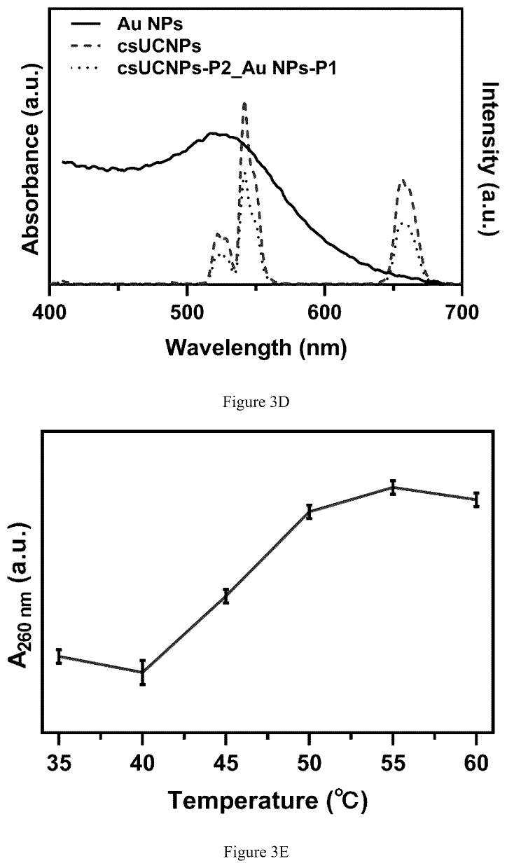

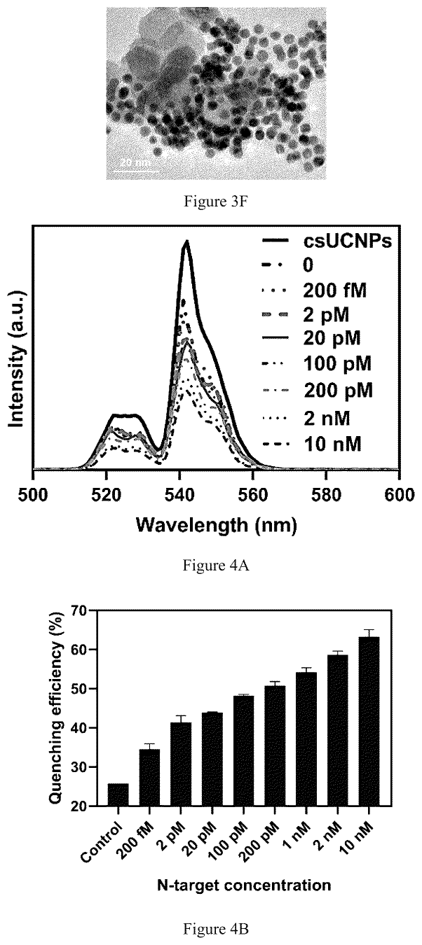

To ensure accurate detection of COVID-19 using the sandwich assay of this invention, methodical control of Au NPs size and DNA adsorption efficiency on Au NPs are important. Based on previous studies, strong luminescence quenching due to resonance energy transfer is preferentially achieved for small gold nanoparticles Thus, a step-by-step protocol was employed for synthesizing small Au NPs via sequential injections of HAuCl₄ precursors. TEM image of Au NPs in FIG. 3A exhibits their uniform size with an average diameter of 4.5 nm, which benefits the quenching efficiency in a donor-to-acceptor FRET-based biosensor. The conjugation of probes plays an important role in hybridization efficiency. Thereby, using a pH-assisted and surfactant-free route instantaneous functionalization of Au NPs with thiolated DNA can be achieved (FIG. 3B). UV-vis spectroscopy was performed to study the surface modification of oligo probe 2 (thiol functionalized oligos and the detailed nucleotides were shown in Table 1). FIG. 3C shows the UV-vis absorption of P2, P2-Au NPs and Au NPs. The intensity at 260 nm was selected as an identification signal to quantify the successful conjugation of probes onto Au NPs. Apart from the surface modification, the surface plasmonic absorption of nanoprobes is crucial since spectral overlapping between donor and acceptor is required for achieving FRET effect. FIG. 3D shows the UV-vis spectrum of Au NPs and upconversion emission of csUCNPs, which overlaps well and allows for high FRET efficiency. The melting characteristics of the assay are vital for determining the temperature in the DNA hybridization. The melting curve in FIG. 3E is plotted from 35 to 60° C. using 20 xi 10 M of N-targets while monitoring the absorption at the wavelength of 260 nm. The melting temperature (Tm) was determined based on half of the percentage drop in the absorption at 260 nm (A₂₆₀), which was conducted at about 45° C. After oligo-modified probes hybridization with target N-gene, the csUCNPs probe is surrounded by the Au NPs probe to form a network structure which is conducive to triggering the FRET effect (FIG. 3F), demonstrating the excellent feasibility of the assay. Upon the completion of the synthetization, modification and feasibility validation, the assay is already primed to specifically target the N gene of COVID-19 and commence viral RNA detection instantly, thereby showing the simplicity of the one-pot viral RNA detection method of this invention without the need for refrained types of reagents and target amplification.

### 5) Detection of N-Target by Upconversion Luminescence Sandwich Assay

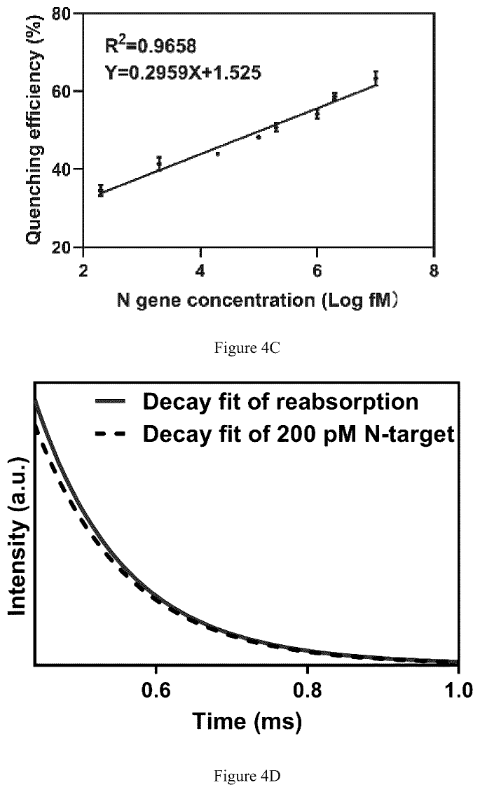

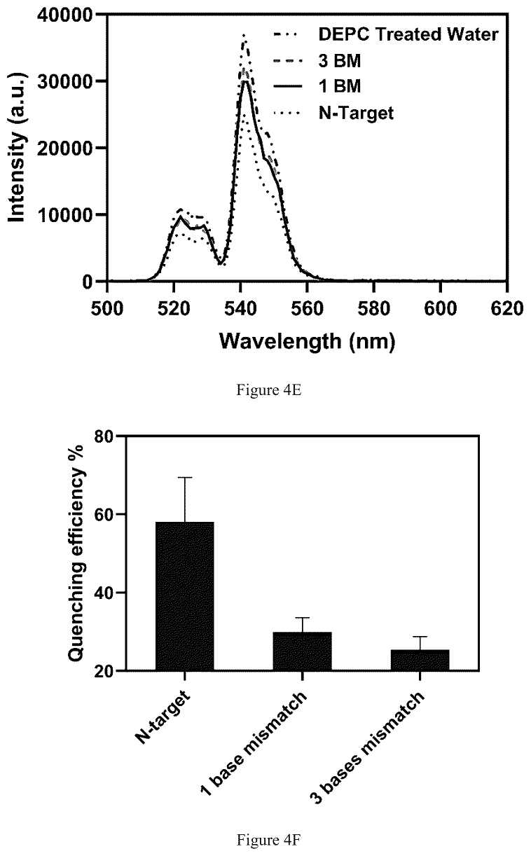

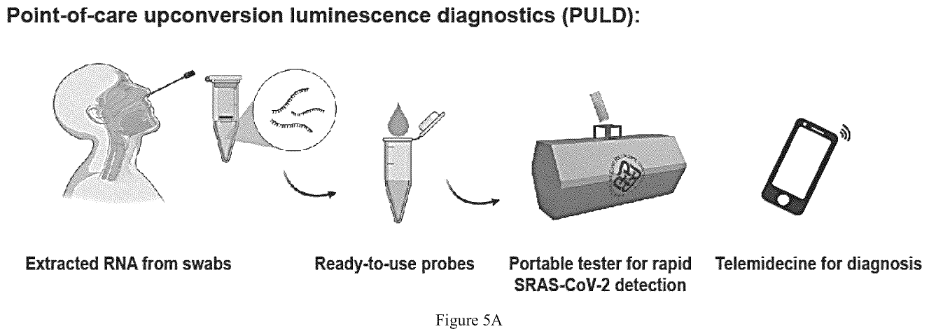

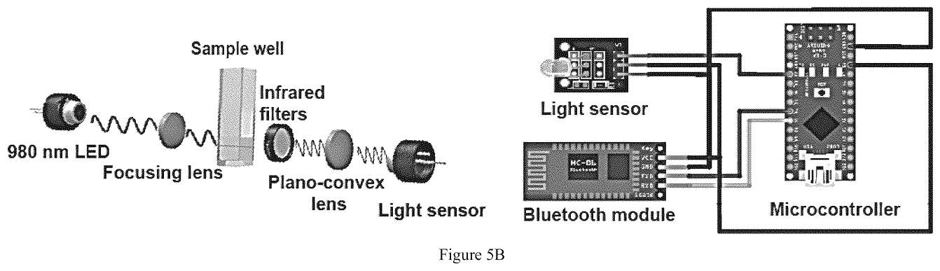

As one of the merits of the sandwich assay of this invention, the superb sensitivity of this rapid and simple virus detection without the burden of target amplification herein was characterized and demonstrated by the analysis of photoluminescence (PL) spectra. Taking advantage of the FRET and strong quenching capability of Au NPs, the PL emission was quenched in accordance with the concentration of N-target increase from 200 fM to 10 nM. As a result of hybridization between N gene targets and the complimentary oligonucleotide probes (UCNPs-P1 and Au NPs-P2), luminescence quenching was realized (FIG. 4A). In addition, the quenching efficiency for each N-target concentration was calculated by the E1 and the enhancement of the efficiency is directly proportional to the amount of N-target (FIG. 4B). FIG. 4C shows a linear response from 10×10⁻⁹ M to 200×10⁻⁵ M as y=0.2959x+1.525. As a result, the control signal plus three times the background signal was used to determine the limit of detection (LOD), which was calculated to be 11.46 fM. The detection limit is much lower than that of the previously published work. Since the luminescence intensity of UCNPs excited at NIR is much higher than that excited at 808 nm, so the quenching can be effectively detected. Moreover, the design and fabrication of the UCNP-Au FRET system (e.g. the size of the nanomaterials and dispersion, surface modification, and the UCNP-Au fluorescence resonance distance) are further optimized in this work, and the LOD correspondingly improved. As compared other detection techniques, COVID-19 screening using lateral flow antigen tests suffers from low specificity and the accessibility of RT-PCR is severely constrained by the need for high-end instruments, multi-step reactions, special reagents, highly skilled personnel in a centralized laboratory, and a long turnaround time. Therefore, the upconversion luminescence diagnostic method is a good alternative as a compromise assay that achieves fast, reliable, ultrasensitive, and easy-to-use point-of-care detection during the pandemic (Table 3). It is worth noting that the plasmonic effect of Au NPs can affect the luminescence intensity of UCNPs by decreasing the luminescence lifetime. In order to further evaluate this effect, the decay of the green emission at 540 nm was investigated. According to FIG. 4D, the lifetime of the green emission was reduced from 128.04 ρs to 118.35 ρs when 200 pM N-target was added. The lifetime reduction is caused by the plasmonic effect upon the existence of the Au NPs and the associated energy transfer. Considering the 520/540 nm emission, energy transfer from the UCNP to the Au NP could provide a non-radiative pathway that competes with the radiative one, resulting in decreasing emission intensity and shortening the lifetime. Since SARS-CoV-2 is experiencing different variations, specificity is paramount for the effectiveness of the assay. Therefore, the specificity of the assay (FIG. 4E and FIG. 4F) was investigated with samples containing 2×10⁻⁹ M of N-target and base mismatch (BM) gene fragments (1 BM and 3 BM). In comparison to the base mismatch group, the N-target group showed a higher quenching efficiency of 55.01%. The initial contact process was not efficient due to one and three base (1 BM and 3 BM) mismatches, resulting in the quenching efficiency by 30.58% and 25.92%, respectively. However, the sensing signals of 1 BM or 3 BM were significantly different from those of the non-targets (DEPC treated water), thus indicating that the assay of this invention is capable of detecting SARS-CoV-2 variants. These characteristics implicate the excellent selectivity of the sandwich assay of this invention.

TABLE 3
A comparison of peer-reviewed articles published on COVID-19 detection technologies.

| | Target | Detection | | Detection | | | | |
|---|---|---|---|---|---|---|---|---|
| Method | analyte | range | LOD | Time | | advantages | limitation | Ref. |
| Naked eye | N-gene | 0.2-3 ng/μL | 0.18 | 10 | min | Equipment- | High false positive | Moitra |
| | | | ng/μl | | | free, Rapid | | et al. |
| Plasmonics | virus | 0.01 pM to | 0.22 pM | ~20 | min | Detection and | The spectrometer is | Qiu et |
| | mRNA | 50 μM | | | | analysis in | relatively | al. |
| | | | | | | real-time | expensive, high | |
| | | | | | | | fabrication cost, | |
| CRISPR, | E gene | 10-2500 | 10 copies | 45 | min | Simple | Risk of | Brough |
| Lateral | and N | copy/μL | per μl | | | process, | contamination | ton et |
| flow, RT- | gene | | | | | low cost | | al. |
| LAMP | | | | | | | | |
| Lateral flow | N-protein | 0-20 ng/mL | 0.65 | 15 | min | Fast, on-site | Low sensitivity | Grant |
| assay | | | ng/mL | | | detection | | et al. |
| RT-PCR | Virus | 10-10000 | 0.15-100 | 120-140 | min | High | Labor-intensive, | Vogels |
| | mRNA | copy/μL | copy/μL | | | throughput, | requires expensive | et al. |
| | | | | | | sensitivity, | equipment and | |
| | | | | | | and | numerous reagents | |
| | | | | | | specificity | to achieve accurate | |
| | | | | | | | results | |
| Upconversion | N gene | 20 fM-200 | 11.46 fM | 20 | min | On site | Lower sensitivity | This |

| | | | | |
|---|---|---|---|---|
| luminescence | pM | detection, | compared to PCR | work |
| diagnostic | | rapid, simple | | |
| | | operation | | |
| | | step, low- | | |
| | | cost device | | |

1. Moitra et al., ACS Nano, 2020, 14(6): 7617-7627.
2. Qiu et al., ACS Nano, 2020. 14, 5268-5277.
3. Broughton et al., Nat. Biotechnol., 2020, 38, 870-874.
4. Grant et al., Anal. Chem., 2020, 92, 11305-11309.
5. Vogels et al., Nat. Microbiol., 2020, 5, 1299-1305.

### 6) PULD Screening SARS-CoV-2 Omicron (B.1.1.529) Variant

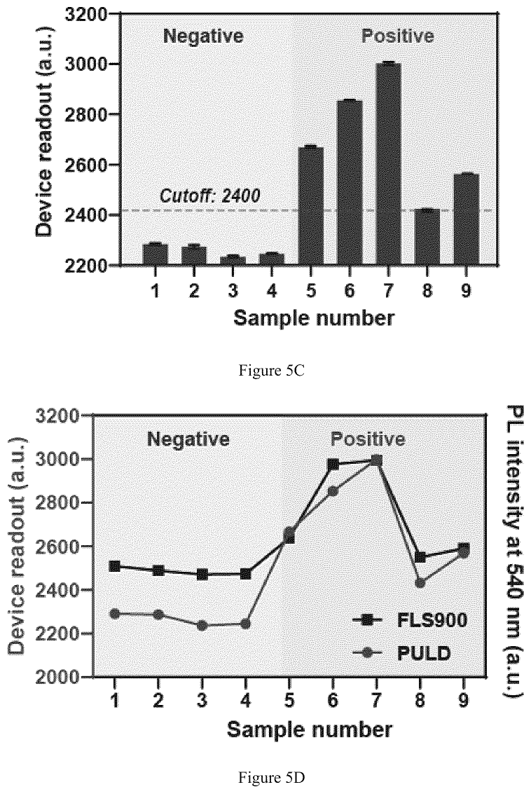

As aforementioned, SARS-CoV-2 RNA was extracted from the specimens and added to the ready-to-use assay, where hybridization was occurred between the oligonucleotide-modified csUCNPs and Au NPs probes and therefore, Au NPs absorbed the fluorescent emission from the UCNPs. This scheme based on the upconversion luminescence quenching is able to produce sensitive and reliable results as compared to other fluorescent dye-based nucleic acid detection techniques. Moreover, due to the simple detection workflow, a PULD system that enables rapid detection of viral RNA and on-board data processing with mobile displaying notification capability was thereby conceptualized and demonstrated (FIG. 5A). Despite its compact size and lightweight, the device integrates all the components needed for the detection of SARS-CoV-2. In particular, the device includes a light source for irradiating UCNP, two bandpass filters, and a highly sensitive light sensor to detect fluorescence-quenching signals. (FIG. 5B). The microcontroller records the collected signals in its memory while simultaneously transmitting them to the user's smartphone via Bluetooth. An APP accompany the PULD was developed to realize a high throughput, facile, simple diagnosis with instantaneous wireless data transmission to a mobile user interface. This APP adopted a simple graphic user interface (GUI) and notifies the user with the diagnostic results by an instinctive graphical representation. It is worth mentioning that the sensitivity of the light sensor is significantly higher than that of conventional photodiode. Briefly, the light sensor of this invention is comprised of a photodiode and an operational amplifier integrator. During measurement, the emission intensity from the assay is determined based on the current changes instead of directly recording the magnitude of the photocurrent. Hence, the sensitivity of the sensor of this invention is several magnitudes higher than that of other commercial light sensors. Combining the short processing time of the device with the high sensitivity and reliability of the sandwich assay. PULD provides an ideal point-of-care screening platform with a short sample-to-answer time. Thus, the capabilities of PULD by testing clinical samples of SARS-CoV-2 Omicron variant was evaluated. In total, five PCR-validated SARS-CoV-2 positive samples and four PCR-validated negative samples were employed in this invention. These samples were prepared by mixing DEPC-treated water and lysis buffer with 0.5 μL, 0.8 μL, 1 μL of Omicron viral RNA, respectively, while lysis buffer and DEPC-treated water were used as controls. FIG. 5C shows the waterfall plot of the read-out values from PULD, with a cutoff of 2400. This set point clearly distinguishes the positive samples from the negative COVID-19 ones, giving a 100% concordance result with RT-PCR (Table 4). A standard PL analyzer was also utilized to compare the diagnosis results of the PULD system. The results from PL analyzer and portable PULD device are well correlated, as shown in FIG. 5D.

TABLE 4
The results of clarifying 9 clinical samples into COVID-19
positive and negative cases using the designed PULD,
which were benchmarked to the standard SARS-CoV-2 diagnostic
kit (Biyotime, COVID-19 RT-qPCR kit).

| | Positive | Negative |
|---|---|---|
| Positive | True positive = 4 | False negative = 0 |
| Negative | False negative = 0 | True negative = 5 |

### Example 3

### Methods for Detecting Viral Proteins

### 1) Preparation of AuNRs

The AuNRs were synthesized using a previous method. Typically, seed solution and growth solution were fabricated as below, Seed solution. 1 mL CTAB solution (0.2 M) and 1 mL HAuCl₄ solution (0.5 mM), after vigorously stirring, 0.2 mL NaBH₄ (0.006 M) was added to the solution and agitated at 1400 rpm for 2 min, a brownish yellow solution was formed and aged for 30 min for further use. Growth solution. 0.7 g CTAB (3.7 mM) and 0.1234 g NaOL (4.7 mM) were dissolved in 25 mL distilled water with vigorously stirring at 50° C., the solution was kept at 25° C. after totally dissolution. Subsequently, 2.4 mL AgNO₃ (4 mM) was added to the above solution and kept undisturbed for another 15 min. 25 mL HAuCl₄ (1 mM) was added to the mixture, and the solution changed from golden to colorless at 90 min. Then 0.15 mL concentrated HCl was added to the solution and agitated slowly for another 15 min. 0.125 mL ascorbic acid (64 mM) added to the solution and stirring at 1500 rpm for 30 s. Finally, 40 μL seed solution was added to the growth solution, and the mixture was stirring vigorously for 30 s and kept still for 12 h at 30° C. The AuNRs were separated by centrifuging at 7000 rpm for 30 min.

### 2) Thiolated of Anti-SARS-COV19 IgM

0.1 mL of anti-SARS-COV19 Immunoglobulin M (IgM, 0.1 mg mL⁻¹) was dissolved in Buph PBS (0.1 M PBS containing 10 mM EDTA). Subsequently, 5 IL DTT (1 M) in Buph PBS were added to the above solution and stirring at 37° C. for 2 h. To remove the abundant DTT, the solution was flushed the Nap-5 column

3) Functionalization of AuNRs with Thiolated Antibodies

Briefly, 10, 50, and 100 μL of thiolated anti-SARS-COV19 IgM (100 1 g/mL) was added dropwise to 1 mL of AuNR solution, respectively. In detail, there was ˜1.2×10¹¹ antibodies in every 10 μL antibody, and the concentration of AuNRs is calculated to be ˜1×10¹¹ in the reaction solution. After stirring for 30 min, 150 μL Methoxy-PEG-SH (PEG-SH) (1 M) was added by drop to the above solution and kept stirring for 2 h at room temperature, the excess PEG-SH was removed by centrifugation colloidal solution after 5 min of GNRs and anti-IgG incubation at room temperature with stirring, which was then kept for 2 h. The optimal concentration of IgM modified Au-IgG NPs were selected by the sensitivity.

### 4) Detection of S Protein in Swab

Different concentrations (1, 2, 4, 8, 16 fg mL⁻¹) of S protein were added to swabs, their emission wavelength were recorded and the Qe at 480 nm and 800 nm were calculated respectively. In blind samples, 2.5, 3.0, 4.5, 5.0, 6.0, 7.5, 8.0, 10.5, and 12 fg S protein were added randomly to the swabs, and the samples were detected at 800 nm to calculate their contents.

### Example 4

### Detection of Viral Proteins

### 1) Design of FRET-Based S Protein Detection Nanoprobe

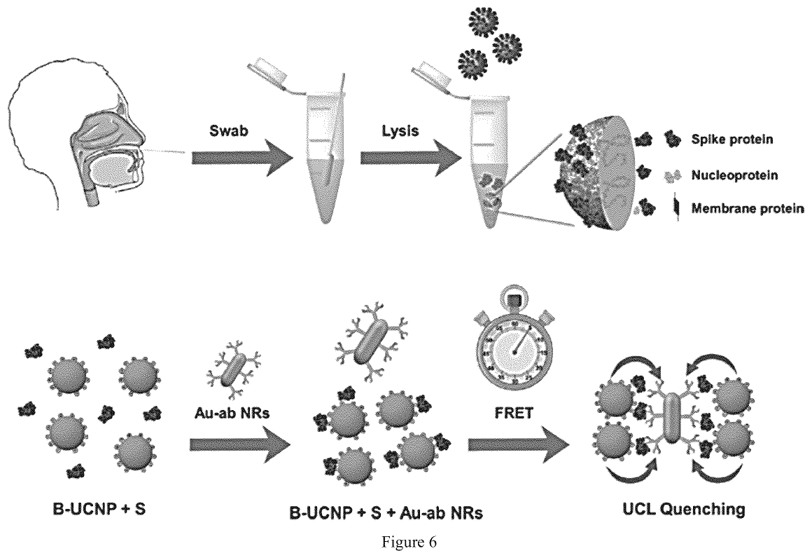

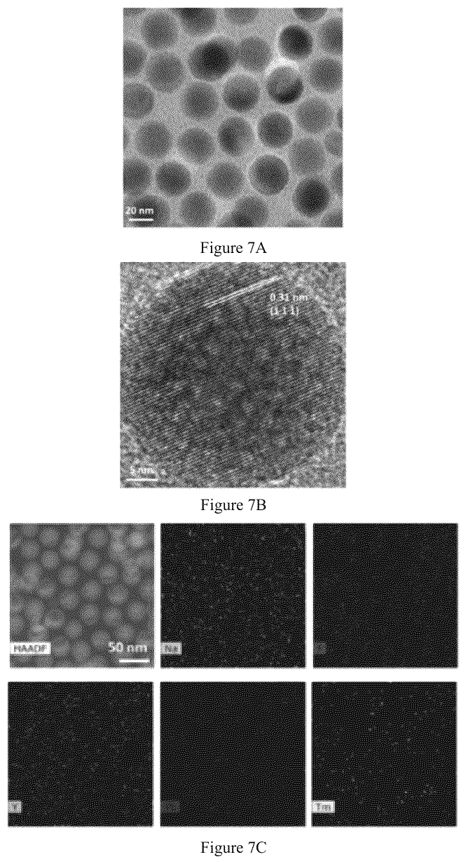

The design and scheme of the detection is illustrated in FIG. 6, in which AuNR-UCNP assemblies are designed to detect S protein with ultralow LOD (1.06 fg mL⁻¹). The formed coresatellite geometric structure of AuNR-UCNP assemblies makes the ultrasensitive detection possible because of the quick combination of UCNPs and AuNRs and the strong resonance between the excited states of UCNPs and AuNRs. Briefly, the swabs containing SARS-CoV-2 are lysed in lysis buffer, the mixture containing negative S protein is firstly co-cultured with the bare UCNPs (B-UCNP, with high positive potential), and a protein “corona” is formed on the surface of B-UCNPs due to the electrostatic attraction. Subsequently, the B-UCNPs with S protein are specifically attached to the Au-ab NRs, forming an orderly rearrangement of B-UNCPs around Au-ab NRs. Thus, an enhanced and concentration dependent FRET process between UCNPs and AuNRs occurs, which induces a significant fluorescence quenching of UCNPs. Moreover, the acid-treated UCNPs may cause a redistribution of Tm on the surface of UCNPs and exhibit high FRET efficiency, leading to ultra-sensitive detection of S protein.

### 2) Characterizations of UCNPs and AuNRs

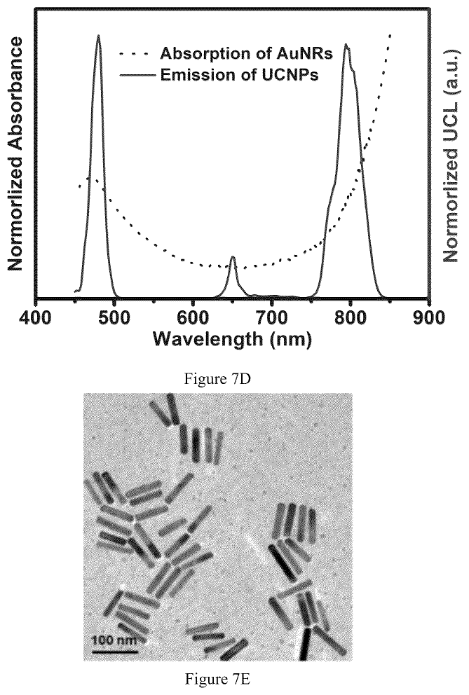

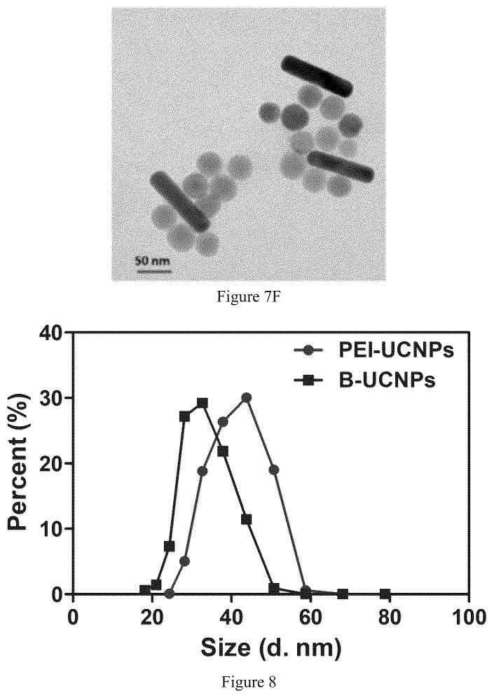

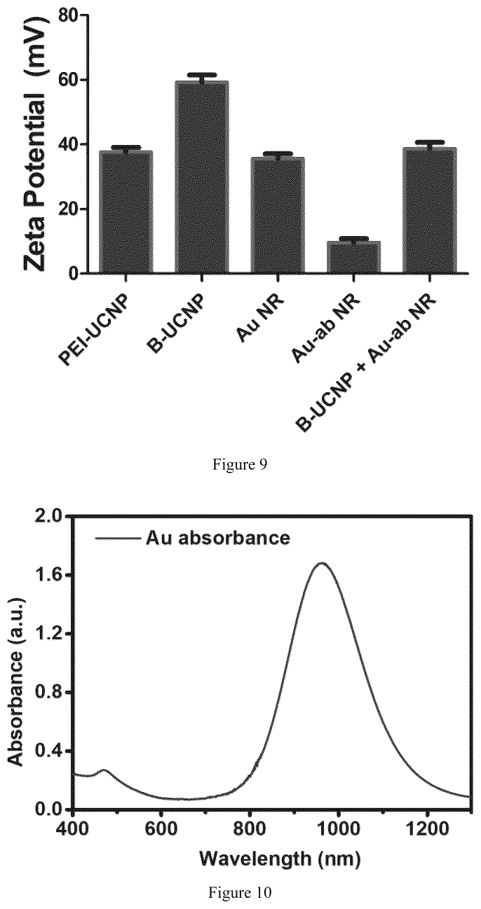

The monodisperse NaYF₄:Yb/Tm NPs were synthesized by a one-step hydrothermal method. Branched PEI was employed as ligand and surfactant to control the size, growth, and photoluminescence (PL) of the UCNPs. As could be seen in FIG. 7, the PEI-NaYF₄:Yb/Tm NPs exhibit excellent water dispersity, with an average size of 30±2 nm. Moreover, the high-resolution STEM image (FIG. 7B) is in accordance with the (111) main plane of XRD results, confirming the cubic structure of PEI-NaYF₄:Yb/Tm. Elemental mapping (FIG. 7C) further illustrates the elemental compositions of Y. Yb, and Tm in UCNPs. The PEI on the surface endows the UCNPs with high dispersity and zeta potential (+37 mV, FIG. 8). On the other hand, the prepared UCNPs with PEI coatings may reduce those emitting ions near the surface of NPs and enlarge the distance between donors and acceptors, thus finally limiting the FRET efficiency. Typically, PEI on the surface of UCNPs efficiently enhance their hydrophilic radius. PEI is estimated to be above 100 nm in its free state. PEI-UCNPs were treated with HCl to remove PEI ligand on the surface and the bare UCNPs were prepared, termed as B-UCNPs. Notably, the average sizes measured by dynamic light scattering (DLS) decrease from 45 nm to 30 nm (FIG. 8). Meanwhile, the zeta potential increases to 60 mV (FIG. 9) after acid treatment and a more stable solution is formed. Thus, most emitting ions are inclined to appear near the surface of the UCNPs and the donors are close to the acceptors. In addition, there is no extra modification on the surface of B-UCNPs, i.e., the donor-to-acceptor distance is minimum (FIG. 8)]. Upon the excitation under NIR laser, the BUCNPs exhibited typical Tm3+ emission peaks (FIG. 7D), the emissions at 480 nm and 650 nm are attributed to the energy transitions from ¹G₄ to ³H₆ and ¹G₄ to ³F₄, respectively. And the emission at 800 nm is arisen from the energy transition from ³H₄ to ³H₆. These main peaks are attributed to blue, red and NIR emissions, which correspond well to the absorption spectra of AuNRs (FIG. 7D).

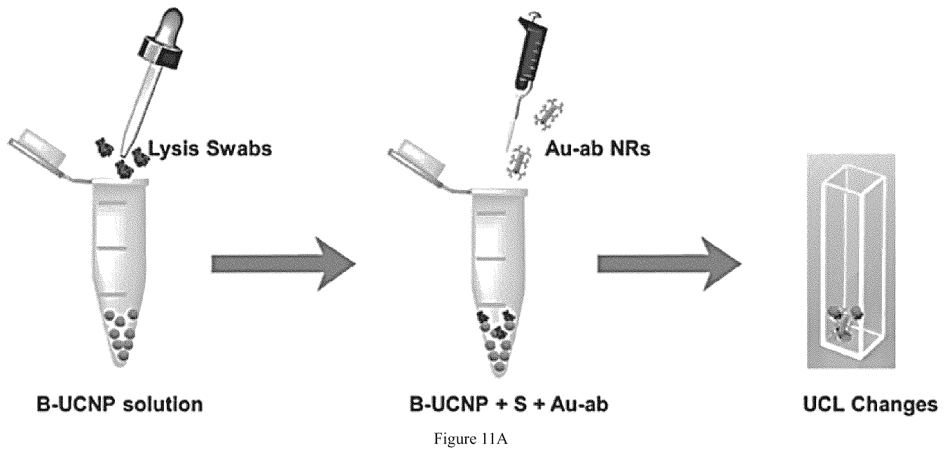

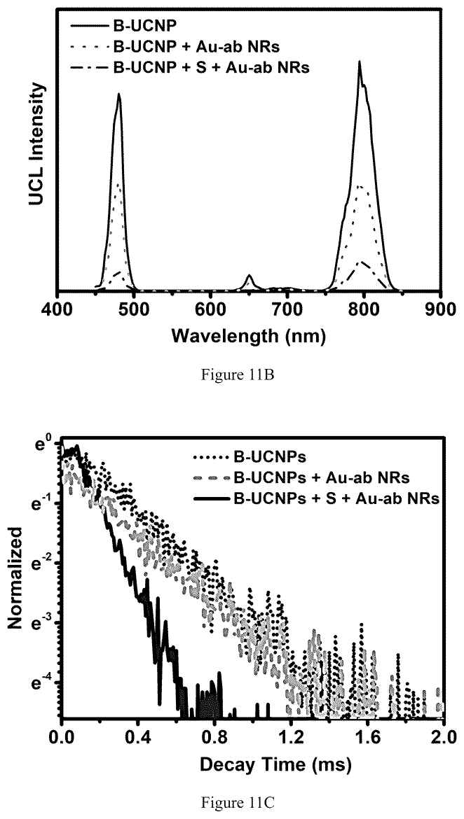

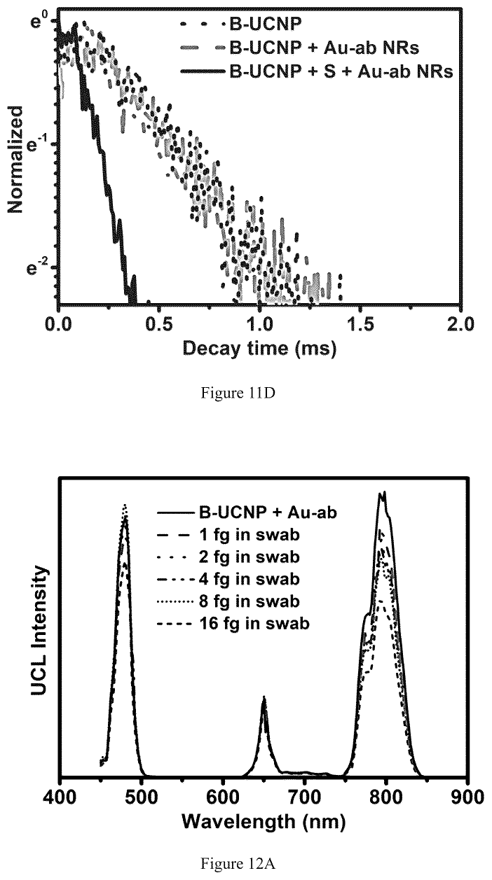

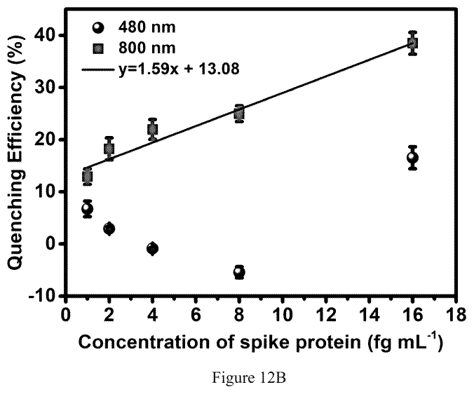

Simultaneously, AuNRs with a length of 78 f 2 nm and width of 15.5 nm, and the aspect ratio of 5.03 were precisely synthesized (FIG. 7E), the longitudinal absorption of the AuNR is centered at 965 nm (FIG. 10), the absorption spectra overlap well with the emission spectra of the UCNPs (480 nm, 800 nm) and the excitation wavelength (FIG. 7D). Here, AuNRs exhibited a high zeta potential (+38 mV) because of the hexadecyl trimethyl ammonium bromide (CTAB) ligand on their surface (FIG. 8).

### 3) FRET-Based Spike Protein Detection

Before further optimization, the validity and accuracy of the nanoprobes was firstly tested. AuNRs were modified with thiolated anti-spike antibody (i.e. human IgM in this case) and further blocked with SH-PEG. Here, the covalent bond of antibody and PEG on the surface of AuNRs is stronger than the ionic bond of CTAB. The successful modification is verified by UV-vis (FIG. 10), the additional absorption peak at 260 nm confirms the successful conjunction of antibodies on the surface of AuNRs. Correspondingly, the zeta potential decrease from 32.8 mV to 10 mV after modification of antibody and PEG (FIG. 8). The positive zeta potential keeps the stabilization of Au-ab solution as well as maintains a certain distance from the high positive UCNPs (FIG. 8), ensuring the stability and specificity of the detection system. Notably, AuNR-UCNP satellite structures are formed after addition of S protein (FIG. 7E). Interestingly, the average distance from UCNP to AuNRs is estimated to be 3.03±0.23 nm according to the TEM images taken from randomly selected locations. It is worth stressing that, the length of S protein at UCNP terminal and antibody at Au surface is larger than the distance in the TEM image because of that their distance has been largely decreased from the dry state characteristic and the hybridization of antigen-antibody effect in TEM image. In a control group with only AuNRs bearing free antibody, no assemblies of Au-UCNP were formed, the UCNPs and AuNRs are in a far distance (>10 nm) and this random distribution is due to the electrostatic repulsive and capillary forces as well.

As shown in FIG. 11A, S protein was firstly incubated with B-UCNPs, then Au-ab NRs were added to the solution and AuNR-UCNP clusters were formed via antigen-antibody reaction, resulting in a close distance between UCNPs and Au-ab NRs, which leads to the decrease of UCL due to FRET effect. The strong absorption at NIR of AuNRs results in the decreased energy and reverse energy transfer to UCNPs, thus finally quenching the luminescence of UCNPs (FIG. 11B). After addition of the target S protein, a specific capture and conjugation of B-UCNP and Au-ab NRs were established, the distance between AuNRs and B-UCNPs decreases significantly via the hybridization of antigen and antibody, which further induce a dramatic UCL quenching at the characteristic emission peaks (FIG. 11B). The energy transfer (ET) efficiency can be expressed as:

$${E}_{T} = \frac{{I}_{C} - {I}_{S}}{{I}_{C}} \qquad ( 5 )$$

I_{c} and Iₛ are the UCL intensities of UCNP and UCNP+AuNRs, respectively.

The E_{T} transfer efficiency (0.51) from UCNP to AuNRs at 800 nm is higher than that (0.45) at 480 nm. In addition, the contribution of Au-ab NRs and S protein in FRET has also been studied using the changes in decay time. As shown in FIG. 11C and FIG. 11D, there is negligible changes of decay time after adding Au-ab NRs to the B-UCNP nanosystem, illustrating non-FRET effect and reabsorption effect in this process. Interestingly, the decay curves of B-UCNPs, B-UCNPs+Au-abs, and B-UCNPs+S+Au-abs at 480 nm are shown in FIG. 11C, the lifetime is reduced from 0.370 ms to 0.212 ms followed by adding S protein in the nanoprobes, and the phenomena is particularly obvious at 800 nm (FIG. 11D), with a lifetime of 0.559 ms and 0.195 ms. Both the decreased UCL intensity and shortened lifetime prove the close distance between the donors and acceptors, and this further induces a strong nonradiative energy transfer from UCNPs to acceptors, which was in accordance with the TEM result (FIG. 7C) and previous studies. Notably, the UCNP-AuNR nanoprobes exhibited a more dramatically decreased in decay time and higher Eᵣ transfer efficiency after addition of S protein at 800 nm compared with 480 nm, illustrating a highly specific and sensitive recognition of target at NIR region.

### 4) Detection of S Protein in Swabs

The collection of saliva samples by patients themselves alleviates demands for supplies of swabs and personal protective equipment. Here, to mimic the clinical detection, the S protein in swabs was tested. As shown in FIG. 12A and FIG. 12B, there is a linear response to different concentration of S protein at 800 nm. By contrast, the response at 480 nm is random and disorganized. Moreover, 10 samples with blind and random concentrations of S protein have further tested for 5 consecutive times, the recoveries and variations (CV %) were shown in Table 5, the recovery rate is between 80%-120%, and the CV % is below 20%, illustrating the reliability of the detection. Therefore, by virtue of the intriguing merits of high stability, non-autofluorescence and low sample interferents, it is important to note that the detection sensitivity and accuracy are highly dependent on the NIR emission (800 nm) of UCNPs.

TABLE 5
Recoveries and variations of S protein
in lysis swab (n = 5 for each sample)

| | S protein level | Mean ± SD | Recovery | CV |
|---|---|---|---|---|
| Sample | (fg mL⁻¹) | (fg mL⁻¹) | (%) | (%) |
| 1 | 2.5 | 2.4 ± 0.3 | 96.0 | 12.5 |
| 2 | 3.0 | 3.2 ± 0.5 | 106.0 | 15.6 |
| 3 | 4.0 | 3.5 ± 0.6 | 87.5 | 17.1 |
| 4 | 5.0 | 6 ± 0.5 | 120.0 | 8.3 |
| 5 | 4.5 | 4.2 ± 0.5 | 93.3 | 11.9 |
| 6 | 6.0 | 6 ± 0.3 | 100.0 | 5.0 |
| 7 | 8.0 | 8.5 ± 1 | 106.3 | 11.8 |
| 8 | 7.5 | 8.1 ± 0.8 | 108.0 | 9.8 |
| 9 | 10.5 | 12 ± 1.2 | 114.0 | 10.0 |
| 10 | 12.0 | 12 ± 0.5 | 100.0 | 4.1 |

### REFERENCES

1. Tsang, M.-K.; Ye, W.; Wang, G.; Li, J.; Yang, M.; Hao. J., Ultrasensitive detection of Ebola virus oligonucleotide based on upconversion nanoprobe/nanoporous membrane system. ACS Nano 2016, 10 (1), 598-605.
2. Yi, Z.; Luo, Z.; Qin, X.; Chen, Q.; Liu, X., Lanthanide-activated nanoparticles: a toolbox for bioimaging, therapeutics, and neuromodulation. Acc. Chem. Res. 2020, 53 (11), 2692-2704.
3. Li, Z.; Yuan, H.; Yuan. W.; Su. Q.; Li, F., Upconversion nanoprobes for biodetections. Coord. Chem. Rev. 2018, 354, 155-168.
4. Gong, Y.; Zheng, Y.; Jin, B.; You, M.; Wang, J.; Li, X.; Lin, M.; Xu, F.; Li, F., A portable and universal upconversion nanoparticle-based lateral flow assay platform for point-of-care testing. Talanta 2019, 201, 126-133.
5. He, H.; Liu, B.; Wen, S.; Liao. J.; Lin. G.; Zhou, J.; in, D., Quantitative lateral flow strip sensor using highly doped upconversion nanoparticles. Anal. Chem. 2018, 90 (21), 12356-12360.
6. L. Ye, X. Xu, S. Song, L. Xu, H. Kuang, C. Xu, Rapid colloidal gold immunochromatographic assay for the detection of SARS-CoV-2 total antibodies after vaccination, J. Mater. Chem. B 2022, 10 (11),1786-1794.
7. F. Ghorbanizamani, H. Moulahoum, F. Zihnioglu, S. Evran, C. Cicek, R. Sertoz, B. Arda, T. Goksel, K. Turhan, S. Timur, Quantitative paper-based dot blot assay for spike protein detection using fuchsine dye-loaded polymersomes, Biosens. Bioelectron. 2021,192, 13484.
8. H. A. Alhadrami, A. M. Hassan. R. Chinnappan, H. AI-Hadrami, W. H. Abdulaal, E. I. Azhar, M. Zourob. Peptide substrate screening for the diagnosis of SARS-CoV-2 using fluorescence resonance energy transfer (FRET) assay, Microchim. Acta 188 2021, (4) 137.
9. Y. Liu, D. Tu, H. Zhu, E. Ma, X. Chen, Lanthanide-doped luminescent nanobioprobes: from fundamentals to biodetection, Nanoscale 2013, 5 (4)1369-1384.
10. Y. Kuang, J. Xu, C. Wang, T. Li, S. Gai, F. He, P. Yang, J. Lin, Fine-tuning Ho-based red-upconversion luminescence by altering NaHoF₄ core size and NaYbF₄ shell thickness, Chem. Mater. 2019, 31 (19) 7898-7909.
11. B. Gu, Q. Zhang, Recent advances on functionalized upconversion nanoparticles for detection of small molecules and ions in biosystems. Adv. Sci. 2018, 5 (3) 1700609.
12. L. Yang, X. Chen, P. Ma, D. Jin, J. Zhou, H. He, Z. Cheng, J. Lin, Upconversion nanoparticles coated with molecularly imprinted polymers for specific sensing, Dalton Trans. 2020, 49 (47) 17200-17206.
13. Wang, F.; Deng, R.; Liu, X., Preparation of core-shell NaGdF₄ nanoparticles doped with luminescent lanthanide ions to be used as upconversion-based probes. Nat. Protoc. 2014, 9 (7), 1634-1644.
14. Dong, H.; Sun, L.-D.; Yan, C.-H., Energy transfer in lanthanide upconversion studies for extended optical applications. Chem. Soc. Rev. 2015, 44 (6), 1608-1634.
15. Chen, G.; Qiu, H.; Prasad, P. N.; Chen, X., Upconversion nanoparticles: design, nanochemistry, and applications in theranostics. Chem. Rev. 2014, 114 (10), 5161-5214.
16. Ge, H.; Wang, D.; Pan. Y.; Guo, Y.; Li, H.; Zhang, F.; Zhu, X.; Li, Y.; Zhang, C.; Huang, L., Sequence-dependent DNA functionalization of upconversion nanoparticles and their programmable assemblies. Angew. Chem. Int. Ed. 2020, 59 (21), 8133-8137.
17. Mendez-Gonzalez, D.; Melle, S.; Calderón, O. G.; Laurenti, M.; Cabrera-Granado, E.; Egatz-Gómez, A.; López-Cabarcos, E.; Rubio-Retama, J.; Diaz, E., Control of upconversion luminescence by gold nanoparticle size: from quenching to enhancement. Nanoscale 2019, 11 (29), 13832-13844.
18. Piella, J.; Bastus, N. G.; Puntes, V., Size-Controlled Synthesis of Sub-10-nanometer Citrate-Stabilized Gold Nanoparticles and Related Optical Properties. Chem. Mater. 2016, 28 (4), 1066-1075.
19. Liu, S.; Chen, G.; Ohulchansky, T. Y.; Swihart, M. T.; Prasad, P. N., Facile synthesis and potential bioimaging applications of hybrid upconverting and plasmonic NaGdF₄:Yb³⁺, Er³⁺/silica/gold nanoparticles. Theranostics 2013, 3 (4), 275.
20. Moitra, P.; Alafeef, M.; Dighe, K.; Frieman, M. B.; Pan, D., Selective naked-eye detection of SARS-CoV-2 mediated by N gene targeted antisense oligonucleotide capped plasmonic nanoparticles. ACS nano 2020, 14 (6), 7617-7627.
21. J. Xu, S. Zhou, D. Tu, W. Zheng, P. Huang, R. Li, Z. Chen, M. Huang, X. Chen, Sub5 nm lanthanide-doped lutetium oxyfluoride nanoprobes for ultrasensitive detection of prostate specific antigen, Chem. Sci. 2016, 7 (4) 2572-2578.
22. X. Ye, L. Jin, H. Caglayan, J. Chen, G. Xing, C. Zheng, V. Doan-Nguyen, Y. Kang, N. Engheta, C. R. Kagan, C. B. Murray, Improved Size-Tunable Synthesis of Monodisperse Gold Nanorods through the Use of Aromatic Additives, ACS Nano 2012, 6 (3), 2804-2817.
23. P. Huang, W. Zheng, S. Zhou. D. Tu, Z. Chen, H. Zhu, R. Li, E. Ma, M. Huang, X. Chen, Lanthanide-doped LiLuF₄ upconversion nanoprobes for the detection of disease biomarkers, Angew. Chem. Int. Edit. 2014, 126. (5) 1276-1281.
24. Z. Dai, T. Gjetting, M. A. Mattebjerg, C. Wu, T. L. Andresen, Elucidating the interplay between DNA-condensing and free polycations in gene transfection through a mechanistic study of linear and branched PEI, Biomaterials 2011, 32 (33) 8626-8634.
25. R. Huschka, J. Zuloaga, M. W. Knight, L. V. Brown, P. Nordlander, N. J. Halas, Light-Induced Release of DNA from Gold Nanoparticles: Nanoshells and Nanorods, J. Am. Chem. Soc. 2011, 133 (31) 12247-12255.
26. J. Shi, F. Tian, J. Lyu, M. Yang, Nanoparticle based fluorescence resonance energy transfer (FRET) for biosensing applications, J. Maier. Chem. B 2015, 3 (35) 6989-7005.
27. F. Vetrone, V. Mahalingam, J. A. Capobianco, Near-infrared-to-blue upconversion in colloidal BaYF₅: Tm³⁺, Yb³⁺ nanocrystals, Chem. Mater. 2009, 2/(9)1847-1851.

What is claimed is:

1) A portable device for detection of a target nucleic acid sequence or target protein in a sample, comprising:
a. A sample chamber for holding a cuvette with a UCNP-based assay and said sample;
b. A NIR light source to initiate fluorescence emission from said UCNP-based assay;
c. A sensor for measuring said fluorescence emission from said UCNP-based assay comprising a light-to-frequency converter;
d. Optics comprising one or more filters and one or more lens, wherein said optics provides an optical path ensuring only said fluorescence emission from said UCNP-based assay reaches said sensor;
e. A microcontroller for
i. communicating with a controlling device;
ii. determining emission intensity from said UCNP-based assay from frequency of a wavetrain from said sensor to produce detection results and sending said detection results to said controlling device;
iii. implementing control instructions from said controlling device;
wherein said UCNP-based assay is one or more systems selected from the group consisting of:
UCNP surface modified with a first oligo probe and AuNP surface modified with a second oligo probe, wherein said first oligo probe and said second oligo probe are independently complimentary to separate segments on said target nucleic acid sequence that are nearby each other; and
ligand-free UCNP with electrostatic attraction to said target protein and AuNR surface modified with antibodies for said target protein.

2) The portable device of claim 1, wherein said sensor measures said fluorescence emission at around 450 nm, 480 nm, 525 nm, 540 nm, 660 nm, or 800 nm.

3) The portable device of claim 1, wherein said microcontroller communicates with said controlling device using one or more wireless technologies selected from the group consisting of Bluetooth, WIFI, RF, and network.

4) The portable device of claim 1, wherein said controlling device is one or more selected from the group consisting of smartphone, computer, and TTL.

5) The portable device of claim 1, wherein said ligand-free UCNP or UCNP surface modified with a first oligo probe is a lanthanide-doped UCNP.

6) The portable device of claim 5, wherein said ligand-free UCNP is NaYF₄:Yb/Tm b-UCNP.

7) The portable device of claim 1, wherein said AuNR comprises:
a. an aspect ratio of around 5; or
b. a longitudinal absorption overlaps with emission spectra of said ligand-free UCNP.

8) The portable device of claim 5, wherein said UCNP surface modified with a first oligo probe is NaGdF₄:Yb/Er@ NaGdF₄ cs-UCNP.

9) The portable device of claim 1, wherein said AuNP surface modified with a second oligo probe has an absorbance maxima overlapping with emission of said UCNP surface modified with a first oligo probe.

10) The portable device of claim 1, wherein said AuNP surface modified with a second oligo probe and said UCNP surface modified with a first oligo probe are spaced less than 10 nm apart after binding to said target nucleic acid sequence.

11) The portable device of claim 1, further comprises a housing for excluding external light from interfering with measurement of said fluorescence emission.

12) The portable device of claim 1, wherein said portable device has a limit-of-detection of about 1 fg mL⁻¹.

13) The portable device of claim 1, wherein
a. said target nucleic acid sequence has a concentration of greater than 11 fM in said sample; or
b. said target protein has a concentration of great than 1 fg mL⁻¹ in said sample.

14) The portable device of claim 1, wherein said target nucleic acid sequence or target gene is obtained from a pathogen.

15) The portable device of claim 14, wherein said pathogen is one or more viruses selected from SARS-CoV-2 virus, Ebola virus, influenza virus, human immunodeficiency virus, and hepatitis virus.

16) The portable device of claim 1, wherein
a. said target nucleic acid sequence is a viral gene selected from nucleocapsid, ORF1a and ORF1b; or
b. said target protein is selected from the group consisting of spike protein, nucleocapsid protein, viral membrane protein, viral envelope protein and antibody against a virus.

17) A method to detect a target nucleic acid sequence or target protein in a sample using the portable device of claim 1, comprising the steps of:
a. Collecting said sample from a subject;
b. Preparing said sample for said UCNP-based assay in a cuvette;
c. Inserting said cuvette into said sample chamber;
d. Initiating fluorescence emission from said UCNP-based assay;
e. Measuring said fluorescence emission using said sensor; and
f. Reading out said detection results from said controlling device.

18) The method of claim 17, wherein said sample is collected from a source selected from the group consisting of nasal swab, oropharyngeal swab, saliva, sputum, urine, blood and feces.

19) The method of claim 17, wherein said step (f) is conducted after 5 to 20 minutes after step (c).

20) The method of claim 17, wherein said fluorescence emission is measured at around 450 nm, 480 nm, 525 nm, 540 nm, 660 nm, or 800 nm at said step (e).


SEQUENCE LISTING (19 sequences)

SEQ ID NO: 1 - RNA, 20 nt, SARS-CoV-2
  source 1..20: mol_type = other RNA
acaccaaaagatcacattgg

SEQ ID NO: 2 - RNA, 20 nt, SARS-CoV-2
  source 1..20: mol_type = other RNA
cccgcaatcctgctaacaat

SEQ ID NO: 3 - RNA, 20 nt, SARS-CoV-2
  source 1..20: mol_type = other RNA
gcatacaatgtaacacaagc

SEQ ID NO: 4 - RNA, 20 nt, SARS-CoV-2
  source 1..20: mol_type = other RNA
cagaacaaacccaaggaaat

SEQ ID NO: 5 - RNA, 20 nt, SARS-CoV-2
  source 1..20: mol_type = other RNA
actgattacaaacattggcc

SEQ ID NO: 6 - DNA, 20 bp, SARS-CoV-2
  source 1..20: mol_type = other DNA
ccaatgtgatcttttggtgt

SEQ ID NO: 7 - DNA, 20 bp, SARS-CoV-2
  source 1..20: mol_type = other DNA
attgttagcaggattgcggg

SEQ ID NO: 8 - DNA, 20 bp, SARS-CoV-2
  source 1..20: mol_type = other DNA
gcttgtgttacattgtatgc

SEQ ID NO: 9 - DNA, 20 bp, SARS-CoV-2
  source 1..20: mol_type = other DNA
atttccttgggtttgttctg

SEQ ID NO: 10 - DNA, 20 bp, SARS-CoV-2
  source 1..20: mol_type = other DNA
ggccaatgtttgtaatcagt

SEQ ID NO: 11 - DNA, 13 bp, unidentified
  misc_feature 1..13: note = Probe 1
  source 1..13: mol_type = other DNA
atttccttggaaa

SEQ ID NO: 12 - DNA, 13 bp, unidentified
  misc_feature 1..13: note = Probe 2
  source 1..13: mol_type = other DNA
aaagtttgttctg

SEQ ID NO: 13 - DNA, 20 bp, unidentified
  misc_feature 1..20: note = 3 BM target
  source 1..20: mol_type = other DNA
cagaagaaacgcaagcaaat

SEQ ID NO: 14 - DNA, 20 bp, unidentified
  misc_feature 1..20: note = 1 BM target
  source 1..20: mol_type = other DNA
cagaagaaacgcaagcaaat

SEQ ID NO: 15 - DNA, 20 bp, unidentified
  misc_feature 1..20: note = N-target
  source 1..20: mol_type = other DNA
cagaacaaacccaaggaaat

SEQ ID NO: 16 - DNA, 22 bp, unidentified
  misc_feature 1..22: note = N-f primer
  source 1..22: mol_type = other DNA
ggggaacttctcctgctagaat

SEQ ID NO: 17 - DNA, 22 bp, unidentified
  misc_feature 1..22: note = N-r primer
  source 1..22: mol_type = other DNA
cagacattttgctctcaagctg

SEQ ID NO: 18 - DNA, 20 bp, unidentified
  misc_feature 1..20: note = RT-qPCR probe
  source 1..20: mol_type = other DNA
ttgctgctgcttgacagatt

SEQ ID NO: 19 - DNA, 99 bp, unidentified
  misc_feature 1..99: note = N-cDNA
  source 1..99: mol_type = other DNA
ggggaacttctcctgctagaatggctggcaatggcggtgatgctgctcttgctttgctgctgcttgacagattgaaccagcttgagagcaaaatgtctg